(19) United States
(12) Patent Application Publication
(10) Pub. No.: US 2024/0336885 A1
(43) Pub. Date: Oct. 10, 2024

(54) UNIFORM CELL CULTURE SUBSTRATE FOR FIXED BED BIOREACTOR

(71) Applicant: CORNING INCORPORATED, CORNING, NY (US)

(72) Inventors: Yujian Sun, Painted Post, NY (US); Vinalia Tjong, Painted Post, NY (US); Yue Zhou, Horseheads, NY (US)

(21) Appl. No.: 18/293,676

(22) PCT Filed: Jul. 21, 2022
(86) PCT No.: PCT/US22/37800

§ 371 Date: Jan. 30, 2024

Related U.S. Application Data
(60) Provisional application No. 63/234,379, filed on Aug. 18, 2021.
(60) Provisional application No. 63/227,693, filed on Jul. 30, 2021.

(51) Int. Cl. C12M 1/12; C12M 1/00

(52) U.S. Cl. CPC C12M 25/18; C12M 25/02; C12M 29/10

(57) ABSTRACT

A bioreactor system for culturing cells is provided having a cell culture vessel with an interior reservoir, and an inlet and outlet connected to the reservoir. The system also includes a cell culture matrix in the reservoir for adhering cells thereto. The cell culture matrix maintains a uniform flow of liquid media through the cell culture matrix, such that the uniform flow satisfies the following expressions: wherein P is greater than or equal to at least one of 0.3, 0.4, 0.5, 0.6, 0.7, 0.8, 0.9, and 0.95, and wherein N is less than or equal to at least one of 0.1, 0.2, 0.3, 0.4, 0.5, 0.6, 0.7, 0.8, 0.9, and 1.0.

### CROSS-REFERENCE TO RELATED APPLICATIONS

This application claims the benefit of priority under 35 U.S.C. § 119 of U.S. Provisional Application Ser. No. 63/234,379 filed on Aug. 18, 2021, the content of which is relied upon and incorporated herein by reference in its entirety.

### FIELD OF THE DISCLOSURE

This disclosure general relates to substrates for culturing cells and fixed bed bioreactors incorporating such substrates, as well as systems and methods for culturing cells. In particular, the present disclosure relates to cell culture substrates an bioreactors with such substrates having uniform fluid flow characteristics.

### BACKGROUND

In the bioprocessing industry, large-scale cultivation of cells is performed for purposes of the production of hormones, enzymes, antibodies, vaccines, and cell therapies. Cell and gene therapy markets are growing rapidly, with promising treatments moving into clinical trials and quickly toward commercialization. However, one cell therapy dose can require billions of cells or trillions of viruses. As such, being able to provide a large quantity of cell products in a short amount of time is critical for clinical success.

A significant portion of the cells used in bioprocessing are anchorage dependent, meaning the cells need a surface to adhere to for growth and functioning. Traditionally, the culturing of adherent cells is performed on two-dimensional (2D) cell-adherent surfaces incorporated in one of a number of vessel formats, such as T-flasks, petri dishes, cell factories, cell stack vessels, roller bottles, and HYPERStack® vessels. These approaches can have significant drawbacks, including the difficulty in achieving cellular density high enough to make it feasible for large scale production of therapies or cells.

Alternative methods have been suggested to increase volumetric density of cultured cells. These include microcarrier cultures performed in stir tanks. In this approach, cells that are attached to the surface of microcarriers are subject to constant shear stress, resulting in a significant impact on proliferation and culture performance. Another example of a high-density cell culture system is a hollow fiber bioreactor, in which cells may form large three-dimensional aggregates as they proliferate in the interspatial fiber space. However, the cells growth and performance are significantly inhibited by the lack nutrients. To mitigate this problem, these bioreactors are made small and are not suitable for large scale manufacturing

Another example of a high-density culture system for anchorage dependent cells is a packed-bed bioreactor system. In this this type of bioreactor, a cell substrate is used to provide a surface for the attachment of adherent cells. Medium is perfused along the surface or through the semi-porous substrate to provide nutrients and oxygen needed for the cell growth. For example, packed bed bioreactor systems that contain a packed bed of support or matrix systems to entrap the cells have been previously disclosed U.S. Pat. Nos. 4,833,083; 5,501,971; and 5,510,262. Packed bed matrices usually are made of porous particles as substrates or non-woven microfibers of polymer. Such bioreactors function as recirculation flow-through bioreactors. One of the significant issues with such bioreactors is the non-uniformity of cell distribution inside the packed bed. For example, the packed bed functions as depth filter with cells predominantly trapped at the inlet regions, resulting in a gradient of cell distribution during the inoculation step. In addition, due to random fiber packaging, flow resistance and cell trapping efficiency of cross sections of the packed bed are not uniform. For example, medium flows fast though the regions with low cell packing density and flows slowly through the regions where resistance is higher due to higher number of entrapped cells. This creates a channeling effect where nutrients and oxygen are delivered more efficiently to regions with lower volumetric cells densities and regions with higher cell densities are being maintained in suboptimal culture conditions.

Another significant drawback of packed bed systems disclosed in a prior art is the inability to efficiently harvest intact viable cells at the end of culture process. Harvesting of cells is important if the end product is cells, or if the bioreactor is being used as part of a “seed train,” where a cell population is grown in one vessel and then transferred to another vessel for further population growth. U.S. Pat. No. 9,273,278 discloses a bioreactor design to improve the efficiency of cell recovery from the packed bed during cells harvesting step. It is based on loosening the packed bed matrix and agitation or stirring of packed bed particles to allow porous matrices to collide and thus detach the cells. However, this approach is laborious and may cause significant cells damage, thus reducing overall cell viability.

An example of a packed-bed bioreactor currently on the market is the iCellis® by produced by Pall Corporation. The iCellis uses small strips of cell substrate material consisting of randomly oriented fibers in a non-woven arrangement. These strips are packed into a vessel to create a packed bed. However, as with similar solutions on the market, there are drawbacks to this type of packed-bed substrate. Specifically, non-uniform packing of the substrate strips creates visible channels within the packed bed, leading to preferential and non-uniform media flow and nutrient distribution through the packed bed. Studies of the iCellis® have noted a “systemic inhomogeneous distribution of cells, with their number increasing from top to bottom of fixed bed,” as well as a “nutrient gradient . . . leading to restricted cell growth and production,” all of which lead to the “unequal distribution of cells [that] may impair transfection efficiency.” (Rational plasmid design and bioprocess optimization to enhance recombinant adeno-associated virus (AAV) productivity in mammalian cells. Biotechnol. J. 2016, 11, 290-297). Studies have noted that agitation of the packed bed may improve dispersion, but would have other drawbacks (i.e., “necessary agitation for better dispersion during inoculation and transfection would induce increased shear stress, in turn leading to reduced cell viability.” Id.). Another study noted of the iCellis® that the uneven distribution of cells makes monitoring of the cell population using biomass sensors difficult (“ . . . if the cells are unevenly distributed, the biomass signal from the cells on the top carriers may not show the general view of the entire bioreactor.” Process Development of Adenoviral Vector Production in Fixed Bed Bioreactor: From Bench to Commercial Scale. Human Gene Therapy, Vol. 26, No. 8, 2015).

In addition, because of the random arrangement of fibers in the substrate strips and the variation in packing of strips between one packed bed and another of a given packed bed reactor system, it can be difficult for users to predict cell culture performance, since the substrate varies between cultures. Furthermore, the packed substrate of many existing bioreactors makes efficiently harvesting cells very difficult or impossible, as it is believed that cells are entrapped by the packed bed.

Existing fixed bed or packed bed bioreactor systems are designed around the concept of a loosely-packed bed, which has been suggested to improve fluid flow. This loose packing results in a low-occupancy bed region (i.e., open spaces are left in the reactor volume due to the reactor not being filled with a substrate to maximum occupancy, let alone having the substrate being compressed into a tightly packed bed).

While manufacturing of viral vectors for early-phase clinical trials is possible with existing platforms, there is a need for a platform that can produce high-quality product in greater numbers in order to reach late-stage commercial manufacturing scale.

There is a need for cell culture substrates and/or matrices, bioreactors, systems, and methods that enable culturing of cells in a high-density format, with uniform cell distribution, and easily attainable and increased harvesting yields. In particular, there is a need for cell culture substates for fixed bed bioreactors that have improved fluid flow characteristics.

### SUMMARY

According to an embodiment of this disclosure, a bioreactor system for culturing cells is provided. The bioreactor system includes a cell culture vessel with at least one interior reservoir, an inlet fluidly connected to the reservoir, and an outlet fluidly connected to the reservoir. The system also includes a cell culture matrix disposed in the reservoir, the cell culture matrix having a substrate for adhering cells thereto. The cell culture matrix is arranged to maintain a uniform flow of liquid media through the cell culture matrix, such that the uniform flow satisfies the following expressions: ∫_{θ1}^{η2}(θ)dθ=P; Δθ=θ₂−θ₁; and Δθ≤N, where P is greater than or equal to at least one of 0.3, 0.4, 0.5, 0.6, 0.7, 0.8, 0.9, and 0.95, and where N is less than or equal to at least one of 0.1, 0.2, 0.3, 0.4, 0.5, 0.6, 0.7, 0.8, 0.9, and 1.0. In embodiments, the substrate is disposed in the reservoir as a packed bed or a fixed bed. In embodiments, N is less than or equal to 0.5, and P is greater than or equal to 0.7. Fluid flow through the cell culture matrix can also satisfy the condition of E(θ) being less than or equal to 0.2 or less than or equal to 0.1, when θ is less than or equal to 0.8, 0.75, 0.7, 0.6, or 0.5. Fluid flow through the cell culture matrix can also satisfy the condition of E(θ) is less than or equal to 0.2 or less than or equal to 0.1, when θ is greater than or equal to 1.2, 1.25, 1.3, 1.4, or 1.5. In embodiments, E′(θ)=0 at a value of θ between 0.6 and 1.4; between 0.7 and 1.3; between 0.8 and 1.2; and between 0.9 and 1.1. The cell culture matrix can be disposed in the interior reservoir at at least 85% occupancy, at least 90% occupancy, at least 95% occupancy, at 100% or greater occupancy, at 102% or greater occupancy, at 105% or greater occupancy, or at 110% or greater occupancy. As an aspect of embodiments, the cell culture matrix is disposed in the interior reservoir in a compressed packing configuration.

One or more embodiments provide a bioreactor system for culturing cells, where the system includes a cell culture vessel with at least one interior reservoir, an inlet fluidly connected to the reservoir, and an outlet fluidly connected to the reservoir. The system further includes a cell culture matrix disposed in the reservoir, the cell culture matrix comprising a substrate for adhering cells thereto, where the cell culture matrix is can maintain a uniform flow of liquid media through the cell culture matrix, such that E(θ) is less than or equal to 0.2 or less than or equal to 0.1, when θ is less than or equal to 0.8, 0.75, 0.7, 0.6, or 0.5. Fluid flow through the cell culture matrix can also satisfy the condition of E(θ) being less than or equal to 0.2 or less than or equal to 0.1, when θ is greater than or equal to 1.2, 1.25, 1.3, 1.4, or 1.5. Fluid flow through the cell culture matrix can also satisfy the condition of E′(θ)=0 at a value of θ between 0.6 and 1.4; between 0.7 and 1.3; between 0.8 and 1.2; and between 0.9 and 1.1. The cell culture matrix can be disposed in the interior reservoir at at least 85% occupancy, at least 90% occupancy, at least 95% occupancy, at 100% or greater occupancy, at 102% or greater occupancy, at 105% or greater occupancy, or at 110% or greater occupancy. As an aspect of embodiments, the cell culture matrix is disposed in the interior reservoir in a compressed packing configuration.

One or more embodiments provide a bioreactor system for culturing cells, where the system includes a cell culture vessel having at least one interior reservoir, an inlet fluidly connected to the reservoir, and an outlet fluidly connected to the reservoir; and a cell culture matrix disposed in the reservoir, the cell culture matrix having a substrate for adhering cells thereto. The cell culture matrix is disposed in the interior reservoir at at least 85% occupancy, at least 90% occupancy, at least 95% occupancy, at 100% or greater occupancy, at 102% or greater occupancy, at 105% or greater occupancy, or at 110% or greater occupancy. As an aspect of embodiments, the cell culture matrix is disposed in the interior reservoir in a compressed packing configuration.

### BRIEF DESCRIPTION OF THE DRAWINGS

FIG. 1A shows a perspective view of a three-dimensional model of a cell culture substrate, according to one or more embodiments of this disclosure.

FIG. 1B is a two-dimensional plan view of the substrate of FIG. 1A.

FIG. 1C is a cross-section along line A-A of the substrate in FIG. 1B.

FIG. 2A shows an example of a cell culture substrate, according to some embodiments.

FIG. 2B shows an example of a cell culture substrate, according to some embodiments.

FIG. 2C shows an example of a cell culture substrate, according to some embodiments.

FIG. 3 shows a schematic view of a cell culture system, according to one or more embodiments.

FIG. 4 shows a schematic view of a cell culture system, according to one or more embodiments.

FIG. 5A shows a plan view of a modeled multi-layer woven mesh cell culture substrate in a tightly packed arrangement, according to one or more embodiments of this disclosure.

FIG. 5B shows a side cross-section view of the multi-layer woven mesh cell culture substrate of FIG. 5A, according to one or more embodiments of this disclosure.

FIG. 6A shows a plan view of a modeled multi-layer woven mesh cell culture substrate in a loosely packed arrangement, according to one or more embodiments of this disclosure.

FIG. 6B shows a side cross-section view of the multi-layer woven mesh cell culture substrate of FIG. 6A, according to one or more embodiments of this disclosure.

FIG. 7A shows the modeled empty space in the dotted-line volume shown in FIGS. 5A and 5B.

FIG. 7B shows the modeled empty space in the dotted-line volume shown in FIGS. 6A and 6B.

FIG. 8A is a photograph of a mesh sample in accordance with mesh sample A from Table 5, according to one or more embodiments of this disclosure.

FIG. 8B is a photograph of a mesh sample in accordance with mesh sample B from Table 5, according to one or more embodiments of this disclosure.

FIG. 8C is a photograph of a mesh sample in accordance with mesh sample C from Table 5, according to one or more embodiments of this disclosure.

FIG. 8D is a photograph of a mesh sample in accordance with mesh sample D from Table 5, according to one or more embodiments of this disclosure.

FIG. 8E is a photograph of a mesh sample in accordance with mesh sample E from Table 5, according to one or more embodiments of this disclosure.

FIG. 8F is a photograph of a mesh sample in accordance with mesh sample F from Table 5, according to one or more embodiments of this disclosure.

FIG. 9 is a bar graph of the permeability of woven mesh samples A-F from FIGS. 8A-8F.

FIG. 10 shows the results of a pressure drop test using samples A-C from FIGS. 8A-C.

FIG. 11A shows a flow uniformity model of a bioreactor with a woven mesh substrate according to one or more embodiments of this disclosure.

FIG. 11B is a close up view of the flow uniformity model of FIG. 11A.

FIG. 12A is a schematic representation of an experimental apparatus for measuring permeability of a non-woven substrate.

FIG. 12B is a schematic representation of an experimental apparatus for measuring permeability of a randomly-packed non-woven substrate.

FIG. 12C is a schematic representation of an experimental apparatus for measuring permeability of an open woven mesh substrate, according to one or more embodiments of this disclosure.

FIG. 13 is a bar graph of the permeability measurements of woven and non-woven cell culture substrates.

FIG. 14A shows simulated flow velocities around a non-woven mesh substrate piece aligned 90° with respect to the flow direction.

FIG. 14B shows simulated flow velocities around a non-woven mesh substrate piece aligned 45° with respect to the flow direction.

FIG. 15A shows simulated flow velocities around a non-woven mesh substrate piece with 1 mm gap between all neighbors.

FIG. 15B shows simulated flow velocities around an open woven mesh with 1 mm gap between all neighbors.

FIG. 16 is a schematic drawing of an experimental setup for measuring residence time distribution of different cell culture substrate samples.

FIG. 17 is a graph showing the change in dye concentration vs. time during residence time distribution measurement for a woven and non-woven cell culture substrate.

FIG. 18 is a graph showing the change in dye concentration vs. normalized volume of flow through a bioreactor having a woven substrate according to embodiments of this disclosure versus other commercially available non-woven cell culture substrates.

FIG. 19A is a photograph of a stack of substrate disks, according to embodiments of this disclosure, at 100% occupancy for an experimental bioreactor,

FIG. 19B is a photograph of a stack of substrate disks, according to embodiments of this disclosure, at 95% occupancy for an experimental bioreactor.

FIG. 20A is a photograph of a bioreactor with a cell culture matrix of stacked layers of substate packed at 95% occupancy with compressed packing, according to one or more embodiments.

FIG. 20B is a photograph of a bioreactor with a cell culture matrix of stacked layers of substate packed at 95% occupancy with loose packing, according to one or more embodiments.

FIG. 21A is a graph of the residence time distribution data from three different packing densities in compress packing configurations, according to one or more embodiments.

FIG. 21B is a graph of the residence time distribution data from three different packing densities in loose packing configurations, according to one or more embodiments.

FIG. 22A is a graph of the residence time distribution data from a fixed bed at 102.5% occupancy at different flow rates, according to one or more embodiments.

FIG. 22B is a graph of the residence time distribution data from a fixed bed at 95% occupancy with compressed packing at different flow rates, according to one or more embodiments.

FIG. 22C is a graph of the residence time distribution data from a fixed bed at 95% occupancy with loose packing at different flow rates, according to one or more embodiments.

FIG. 23A is a graph of the residence time distribution data from fixed beds in various occupancy percentages with compressed packing and the presence of bubbles, according to one or more embodiments.

FIG. 23B is a graph of the residence time distribution data from fixed beds in various occupancy percentages with loose packing and the presence of bubbles, according to one or more embodiments.

### DETAILED DESCRIPTION

Various embodiments of the disclosure will be described in detail with reference to drawings, if any. Reference to various embodiments does not limit the scope of the invention, which is limited only by the scope of the claims attached hereto. Additionally, any examples set forth in this specification are not limiting and merely set forth some of the many possible embodiments of the claimed invention.

Embodiments of this disclosure include a cell culture substrate, as well as cell culture or bioreactor systems incorporating such a substrate, and methods of culturing cells using such a substrate and bioreactor systems. The substrate and bioreactor systems incorporating the same exhibit improved flow characteristics through the substrate. For example, more uniform flow is achieved through the substrate, and non-uniform flow resulting from channeling or turbulent flow is reduced or eliminated. Flow “dead zones” in the substrate or fixed bed of the bioreactor are greatly reduced or eliminated compared to existing solutions. The result is a substrate or fixed bed that allows for uniform perfusion throughout the substrate or fixed bed, which promotes cell health during cell culture and an efficient cell culture process in terms of not only the culturing of cells, but also cell seeding, and harvesting of cells or cell by-products.

In conventional large-scale cell culture bioreactors, different types of fixed bed or packed bed bioreactors have been used. Usually these packed beds contain porous matrices to retain adherent or suspension cells, and to support growth and proliferation. Packed-bed matrices provide high surface area to volume ratios, so cell density can be higher than in the other systems. However, the packed bed often functions as a depth filter, where cells are physically trapped in the entangled fibers of the matrix. Thus, because of linear flow of the cell inoculum through the packed bed, cells are subject to heterogeneous distribution inside the packed bed, leading to variations in cell density through the depth or width of the packed bed. For example, cell density may be higher at the inlet region of a bioreactor and significantly lower nearer to the outlet of the bioreactor. This non-uniform distribution of the cells inside of the packed bed significantly hinders scalability and predictability of such bioreactors in bioprocess manufacturing, and can even lead to reduced efficiency in terms of growth of cells or viral vector production per unit surface area or volume of the packed bed.

Another problem encountered in packed bed bioreactors disclosed in prior art is the channeling effect. Due to random nature of packed nonwoven fibers, the local fiber density at any given cross section of the packed bed is not uniform. Liquid media flows quickly in the regions with low fiber density (high bed permeability) and much slower in the regions of high fiber density (lower bed permeability). The resulting non-uniform media perfusion across the packed bed creates the channeling effect, which manifests itself as significant nutrient and metabolite gradients that negatively impact overall cell culture and bioreactor performance. Cells located in the regions of low media perfusion will starve and very often die from the lack of nutrients or metabolite poisoning. The flow can be so non-uniform that there are effectively flow “dead zones” in the packed bed, where perfusion does not occur and the delivery of any nutrients to cells in those areas is limited to diffusion mechanics in the media.

Cell harvesting is yet another problem encountered when bioreactors packed with non-woven fibrous scaffolds are used. Due to packed-bed functions as depth filter, cells that are released at the end of cell culture process are entrapped inside the packed bed, and cell recovery is very low. This significantly limits utilization of such bioreactors in bioprocesses where live cells are the products or where live cells need to be harvested for further processing to capture cell by-products. Thus, the non-uniformity leads to areas with different exposure to flow and shear, effectively reducing the usable cell culture area, causing non-uniform culture, and interfering with transfection efficiency and cell release.

In addition, while existing solutions suggest that loosely packed beds are preferable, the current disclosure will illustrate the converse: densely packed beds, in accordance with embodiments of the present disclosure, can actually improve performance of a fixed bed for cell culture. For example, embodiments of this disclosure show superior flow uniformity at high packed densities of substrate material in the bioreactor. Embodiments also show a reduction in the presence of bubbles in the fixed bed and the bioreactor system, in general. Nonetheless, even if bubbles are present in the system, the uniform fluid flow characteristics described herein can be maintained. Bubbles are commonly observed during cell cultures and can easily be trapped in fixed bed bioreactors, including the substrates. Once trapped, they disturb flow direction and reduce flow uniformity, therefore interfering with cell seeding and nutrient supply. For cell culture in a fixed bed reactor, higher flow rates are consistently used to maintain oxygen and nutrient supply to support high density cell growth. High flow rates can potentially lift mesh disks in the packed bed if the disks are not restricted. This can change local permeability and disturb flow direction. By using high compression and high occupancy, the disks are restricted in place and the negative impacts from bubbles and high flow rates can be mitigated. The loose packing or shifting of the packed bed can also mean inconsistent packing of packed beds in conventional systems, which can result in variations in permeability and channeling. However, when the bed is compacted according to embodiments of this disclosure, these negative flow characteristics can be avoided.

The above advantages of embodiments of this disclosure are quite unexpected in light of the preference for loosely-packed beds in the marketplace. These improvements also exist in embodiments regardless of fluid flow rate (including, e.g., channeling) or size of the fixed bed or bioreactor. Thus, the advantages are present across a range of bioreactor scales. A further advantage includes reducing or eliminating shifting of the fixed bed or a change in compaction of the fixed bed due to being subjected to various flow rates or due to mechanical forces during handling and/or shipping. In addition, the higher compaction of the fixed bed results in more surface area for cells.

As used herein, “occupancy” is defined as percentage of substrate packed into the bed volume of the reactor, relative to 100% occupancy. To determine 100% occupancy, the space height of the reactor bed space is divided by the thickness of each substrate layer to obtain the number of substrate layers for 100% occupancy. Occupancies of less than 100% (also referred to as loose packing) is obtained by removing the requisite percentage of mesh layers. For example, removing 5% of the layers from the 100% occupancy condition results in 95% occupancy. It is also possible to achieve greater than 100% occupancy. For example, due to some elasticity in the material of a substrate, the bed could be compressed to some extent and thus create additional space for most substate layers. If additional layers are added to that space above the 100% occupancy number of layers, then the resulting occupancy would be greater than 100%. For example, if 2.5% extra layers are added, the occupancy would be 102.5%.

To address these and other problems of existing cell culture solutions, embodiments of the present disclosure provide cell growth substrates, matrices of such substrates, and/or packed-bed systems using such substrates that enable efficient and high-yield cell culturing for anchorage-dependent cells and production of cell products (e.g., proteins, antibodies, viral particles). Embodiments include a porous cell-culture matrix made from an ordered and regular array of porous substrate material that enables uniform cell seeding and media/nutrient perfusion, as well as efficient cell harvesting. Embodiments also enable scalable cell-culture solutions with substrates and bioreactors capable of seeding and growing cells and/or harvesting cell products from a process development scale to a full production size scale, without sacrificing the uniform performance of the embodiments. For example, in some embodiments, a bioreactor can be easily scaled from process development scale to product scale with comparable viral genome per unit surface area of substrate (VG/cm²) across the production scale. The harvestability and scalability of the embodiments herein enable their use in efficient seed trains for growing cell populations at multiple scales on the same cell substrate. In addition, the embodiments herein provide a cell culture matrix having a high surface area that, in combination with the other features described, enables a high yield cell culture solution. In some embodiments, for example, the cell culture substrate and/or bioreactors discussed herein can produce 10¹⁶ to 10¹⁸ viral genomes (VG) per batch.

In one embodiment, a matrix is provided with a structurally defined surface area for adherent cells to attach and proliferate that has good mechanical strength and forms a highly uniform multiplicity of interconnected fluidic networks when assembled in a packed bed or other bioreactor. In particular embodiments, a mechanically stable, non-degradable woven mesh can be used as the substrate to support adherent cell production. The cell culture matrix disclosed herein supports attachment and proliferation of anchorage dependent cells in a high volumetric density format. Uniform cell seeding of such a matrix is achievable, as well as efficient harvesting of cells or other products of the bioreactor. In addition, the embodiments of this disclosure support cell culturing to provide uniform cell distribution during the inoculation step and achieve a confluent monolayer or multilayer of adherent cells on the disclosed matrix, and can avoid formation of large and/or uncontrollable 3D cellular aggregates with limited nutrient diffusion and increased metabolite concentrations. Thus, the matrix eliminates diffusional limitations during operation of the bioreactor. In addition, the matrix enables easy and efficient cell harvest from the bioreactor. The structurally defined matrix of one or more embodiments enables complete cell recovery and consistent cell harvesting from the packed bed of the bioreactor.

According to some embodiments, a method of cell culturing is also provided using bioreactors with the matrix for bioprocessing production of therapeutic proteins, antibodies, viral vaccines, or viral vectors.

In contrast to existing cell culture substrates used in cell culture bioreactors (i.e., non-woven substrates of randomly ordered fibers), embodiments of this disclosure include a cell culture substrate having a defined and ordered structure. The defined and order structure allows for consistent and predictable cell culture results. In addition, the substrate has an open porous structure that prevents cell entrapment and enables uniform flow through the packed bed. This construction enables improved cell seeding, nutrient delivery, cell growth, and cell harvesting. According to one or more particular embodiments, the matrix is formed with a substrate material having a thin, sheet-like construction having first and second sides separated by a relatively small thickness, such that the thickness of the sheet is small relative to the width and/or length of the first and second sides of the substrate. In addition, a plurality of holes or openings are formed through the thickness of the substrate. The substrate material between the openings is of a size and geometry that allows cells to adhere to the surface of the substrate material as if it were approximately a two-dimensional (2D) surface, while also allowing adequate fluid flow around the substrate material and through the openings. In some embodiments, the substrate is a polymer-based material, and can be formed as a molded polymer sheet; a polymer sheet with openings punched through the thickness; a number of filaments that are fused into a mesh-like layer; a 3D-printed substrate; or a plurality of filaments that are woven into a mesh layer. The physical structure of the matrix has a high surface-to-volume ratio for culturing anchorage dependent cells. According to various embodiments, the matrix can be arranged or packed in a bioreactor in certain ways discussed here for uniform cell seeding and growth, uniform media perfusion, and efficient cell harvest.

Embodiments of this disclosure can achieve viral vector platforms of a practical size that can produce viral genomes on the scale of greater than about 10¹⁴ viral genomes per batch, greater than about 10¹⁵ viral genomes per batch, greater than about 10¹⁶ viral genomes per batch, greater than about 10¹⁷ viral genomes per batch, or up to or greater than about g 10¹⁶ viral genomes per batch. In some embodiments, productions is about 10¹⁵ to about 10¹⁸ or more viral genomes per batch. For example, in some embodiments, the viral genome yield can be about 10¹⁵ to about 10¹⁶ viral genomes or batch, or about 10¹⁶ to about 10¹⁹ viral genomes per batch, or about 10¹⁶-10¹⁸ viral genomes per batch, or about 10¹⁷ to about 10¹⁹ viral genomes per batch, or about 10¹⁸ to about 10¹⁹ viral genomes per batch, or about 10¹⁸ or more viral genomes per batch.

In addition, the embodiments disclosed herein enable not only cell attachment and growth to a cell culture substrate, but also the viable harvest of cultured cells. The inability to harvest viable cells is a significant drawback in current platforms, and it leads to difficulty in building and sustaining a sufficient number of cells for production capacity. According to an aspect of embodiments of this disclosure, it is possible to harvest viable cells from the cell culture substrate, including between 80% to 100% viable, or about 85% to about 99% viable, or about 90% to about 99% viable. For example, of the cells that are harvested, at least 80% are viable, at least 85% are viable, at least 90% are viable, at least 91% are viable, at least 92% are viable, at least 93% are viable, at least 94% are viable, at least 95% are viable, at least 96% are viable, at least 97% are viable, at least 98% are viable, or at least 99% are viable. Cells may be released from the cell culture substrate using, for example, trypsin, TrypLE, or Accutase.

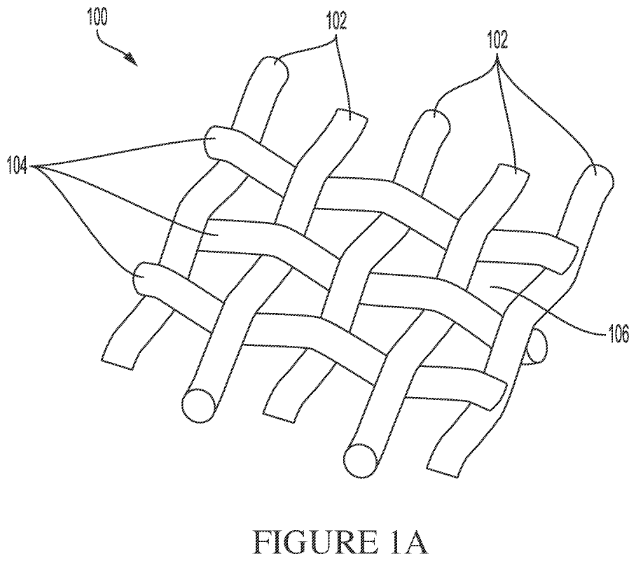

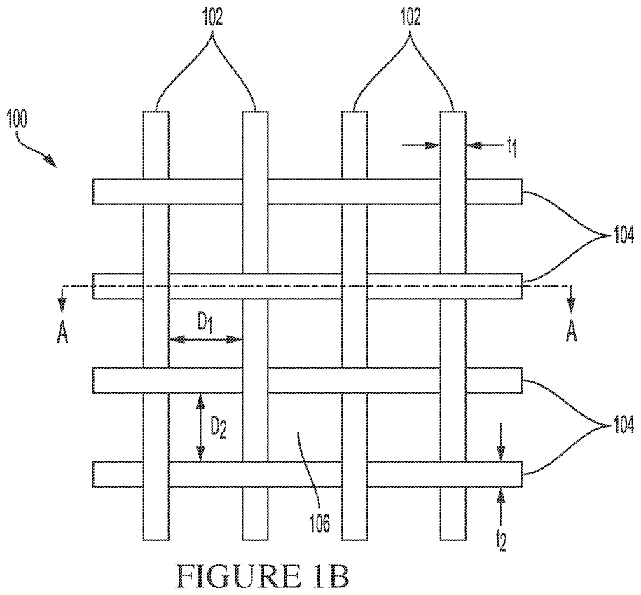

FIGS. 1A and 1B show a three-dimensional (3D) perspective view and a two-dimensional (2D) plan view, respectively, of a cell culture substrate 100, according to an example of one or more embodiments of this disclosure. The cell culture substrate 100 is a woven mesh layer made of a first plurality of fibers 102 running in a first direction and a second plurality of fibers 104 running in a second direction. The woven fibers of the substrate 100 form a plurality of openings 106, which can be defined by one or more widths or diameters (e.g., D₁, D₂). The size and shape of the openings can vary based on the type of weave (e.g., number, shape and size of filaments; angle between intersecting filaments, etc.). A woven mesh may be characterized as, on a macro-scale, a two-dimensional sheet or layer. However, a close inspection of a woven mesh reveals a three-dimensional structure due to the rising and falling of intersecting fibers of the mesh. Thus, as shown in FIG. 1C, a thickness T of the woven mesh 100 may be thicker than the thickness of a single fiber (e.g., t₁). As used herein, the thickness T is the maximum thickness between a first side 108 and a second side 110 of the woven mesh. Without wishing to be bound by theory, it is believed that the three-dimensional structure of the substrate 100 is advantageous as it provides a large surface area for culturing adherent cells, and the structural rigidity of the mesh can provide a consistent and predictable cell culture matrix structure that enables uniform fluid flow.

In FIG. 1B, the openings 106 have a diameter D₁, defined as a distance between opposite fibers 102, and a diameter D₂, defined as a distance between opposite fibers 104. D₁ and D₂ can be equal or unequal, depending on the weave geometry. Where D₁ and D₂ are unequal, the larger can be referred to as the major diameter, and the smaller as the minor diameter. In some embodiments, the diameter of an opening may refer to the widest part of the opening. Unless otherwise specified, the opening diameter, as used herein, will refer to a distance between parallel fibers on opposite sides of an opening.

A given fiber of the plurality of fibers 102 has a thickness t₁, and a given fiber of the plurality of fibers 104 has a thickness t₂. In the case of fibers of round cross-section, as shown in FIG. 1A, or other three-dimensional cross-sections, the thicknesses t₁ and t₂ are the maximum diameters or thicknesses of the fiber cross-section. According to some embodiments, the plurality of fibers 102 all have the same thickness t₁, and the plurality of fiber 104 all have the same thickness t₂. In addition, t₁ and t₂ may be equal. However, in one or more embodiments, t₁ and t₂ are not equal such as when the plurality of fibers 102 are different from the plurality of fiber 104. In addition, each of the plurality of fibers 102 and plurality of fibers 104 may contain fibers of two or more different thicknesses (e.g., t₁ₐ, t_{1b}, etc., and t₂ₐ, t_{2b}, etc.). According to embodiments, the thicknesses t₁ and t₂ are large relative to the size of the cells cultured thereon, so that the fibers provide an approximation of a flat surface from the perspective of the cell, which can enable better cell attachment and growth as compared to some other solutions in which the fiber size is small (e.g., on the scale of the cell diameter). Due to three-dimensional nature of woven mesh, as shown in FIGS. 1A-1C, the 2D surface area of the fibers available for cell attachment and proliferation exceeds the surface area for attachment on an equivalent planar 2D surface.

In one or more embodiments, a fiber may have a diameter in a range of about 50 μm to about 1000 μm; about 100 μm to about 750 μm; about 125 μm to about 600 μm; about 150 μm to about 500 μm; about 200 μm to about 400 μm; about 200 μm to about 300 μm; or about 150 μm to about 300 μm. On a microscale level, due to the scale of the fiber compared to the cells (e.g., the fiber diameters being larger than the cells), the surface of monofilament fiber is presented as an approximation of a 2D surface for adherent cells to attach and proliferate. Fibers can be woven into a mesh with openings ranging from about 100 μm×100 μm to about 1000 μm×1000 μm. In some embodiments, the opening may have a diameter o about 50 μm to about 1000 μm; about 100 μm to about 750 μm; about 125 μm to about 600 μm; about 150 μm to about 500 μm; about 200 μm to about 400 μm; or about 200 μm to about 300 μm. These ranges of the filament diameters and opening diameters are examples of some embodiments, but are not intended to limit the possible feature sizes of the mesh according to all embodiments. The combination of fiber diameter and opening diameter is chosen to provide efficient and uniform fluid flow through the substrate when, for example, the cell culture matrix is comprises a number of adjacent mesh layers (e.g., a stack of individual layers or a rolled mesh layer).

Factors such as the fiber diameter, opening diameter, and weave type/pattern will determine the surface area available for cell attachment and growth. In addition, when the cell culture matrix includes a stack, roll, or other arrangement of overlapping substrate, the packing density of the cell culture matrix will impact the surface area of the packed bed matrix. Packing density can vary with the packing thickness of the substrate material (e.g., the space needed for a layer of the substrate). For example, if a stack of cell culture matrix has a certain height, each layer of the stack can be said to have a packing thickness determined by dividing the total height of the stack by the number of layers in the stack. The packing thickness will vary based on fiber diameter and weave, but can also vary based the alignment of adjacent layers in the stack. For instance, due to the three-dimensional nature of a woven layer, there is a certain amount of interlocking or overlapping that adjacent layers can accommodate based on their alignment with one another. In a first alignment, the adjacent layers can be tightly nestled together, but in a second alignment, the adjacent layers can have zero overlap, such as when the lower-most point of the upper layer is in direct contact with the upper-most point of the lower layer. It may be desirable for certain applications to provide a cell culture matrix with a lower density packing of layers (e.g., when higher permeability is a priority) or a higher density of packing (e.g., when maximizing substrate surface area is a priority). According to one or more embodiments, the packing thickness can be from about 50 μm to about 1000 μm; about 100 μm to about 750 μm; about 125 μm to about 600 μm; about 150 μm to about 500 μm; about 200 μm to about 400 μm; about 200 μm to about 300 μm.

The above structural factors can determine the surface area of a cell culture matrix, whether of a single layer of cell culture substrate or of a cell culture matrix having multiple layers of substrate). For example, in a particular embodiment, a single layer of woven mesh substrate having a circular shape and diameter of 6 cm can have an effective surface area of about 68 cm². The “effective surface area,” as used herein, is the total surface area of fibers in a portion of substrate material that is available for cell attachment and growth. Unless stated otherwise, references to “surface area” refer to this effective surface area. According to one or more embodiments, a single woven mesh substrate layer with a diameter of 6 cm may have an effective surface area of about 50 cm² to about 90 cm²; about 53 cm² to about 81 cm²; about 68 cm²; about 75 cm²; or about 81 cm². These ranges of effective surface area are provided for example only, and some embodiments may have different effective surface areas. The cell culture matrix can also be characterized in terms of porosity, as discussed in the Examples herein.

The substrate mesh can be fabricated from monofilament or multifilament fibers of polymeric materials compatible in cell culture applications, including, for example, polystyrene, polyethylene terephthalate, polycarbonate, polyvinylpyrrolidone, polybutadiene, polyvinylchloride, polyethylene oxide, polypyrroles, and polypropylene oxide. Mesh substrates may have a different patterns or weaves, including, for example knitted, warp-knitted, or woven (e.g., plain weave, twilled weave, dutch weave, five needle weave).

The surface chemistry of the mesh filaments may need to be modified to provide desired cell adhesion properties. Such modifications can be made through the chemical treatment of the polymer material of the mesh or by grafting cell adhesion molecules to the filament surface. Alternatively, meshes can be coated with thin layer of biocompatible hydrogels that demonstrate cell adherence properties, including, for example, collagen or Matrigel®. Alternatively, surfaces of filament fibers of the mesh can be rendered with cell adhesive properties through the treatment processes with various types of plasmas, process gases, and/or chemicals known in the industry. In one or more embodiments, however, the mesh is capable of providing an efficient cell growth surface without surface treatment.

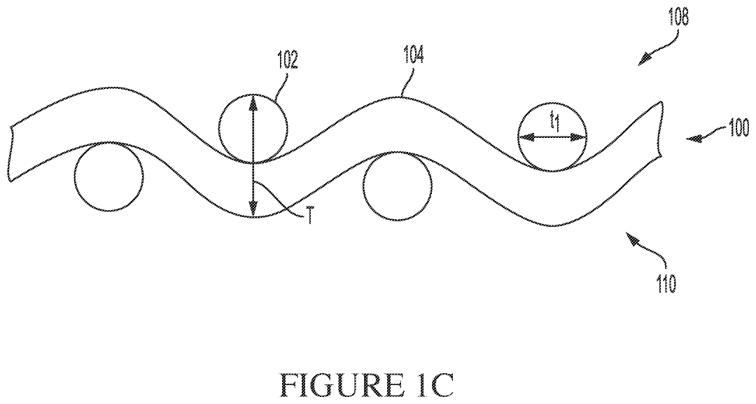

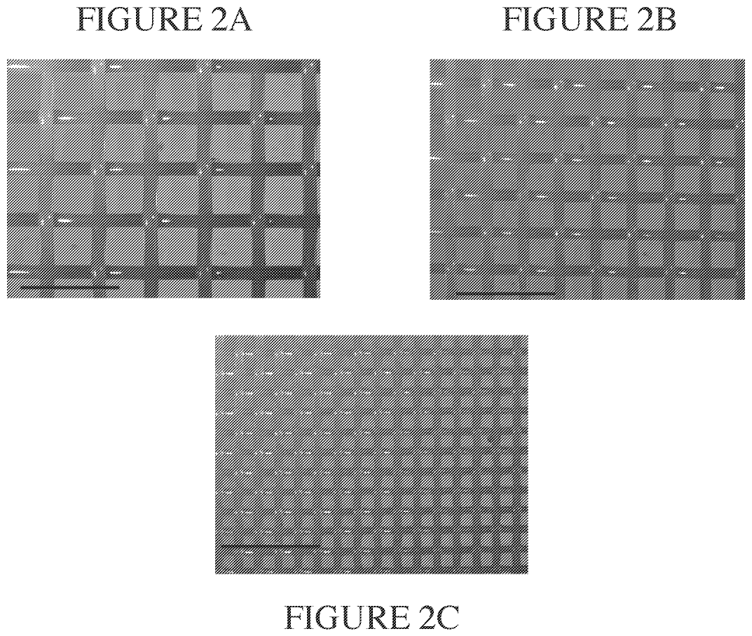

FIGS. 2A-2C show different examples of woven mesh according to some contemplated embodiments of this disclosure. The fiber diameter and opening size of these meshes are summarized in Table 1 below, as well as the approximate magnitude of increase in cell culture surface area provided by a single layer of the respective meshes relative to a comparable 2D surface. In Table 1, Mesh A refers to the mesh of FIG. 2A, Mesh B to the mesh of FIG. 2B, and Mesh C to the mesh of FIG. 2C. The three mesh geometries of Table 1 are examples only, and embodiments of this disclosure are not limited to these specific examples. Because Mesh C offers the highest surface area, it may be advantageous in achieving a high density in cell adhesion and proliferation, and thus provide the most efficient substrate for cell culturing. However, in some embodiments, it may be advantageous for the cell culture matrix to include a mesh with lower surface area, such as Mesh A or Mesh B, or a combination of meshes of different surface areas, to achieve a desired cell distribution or flow characteristics within the culture chamber, for example.

TABLE 1
Comparison of meshes in FIGS. 2A-2C, and the resulting increase
in cell culture surface area as compared to a 2D surface.

| | Mesh A | | Mesh B | | Mesh C | |
|---|---|---|---|---|---|---|
| Fiber diameter | 273 ± 3 | μm | 218 ± 3 | μm | 158 ± 3 | μm |
| Mesh opening | 790 × 790 | μm | 523 × 523 | μm | 244 × 244 | μm |

| | | | |
|---|---|---|---|
| Surface area | ×1.6 | ×1.8 | ×2.5 |
| increase of one | | | |
| layer of mesh | | | |
| compared to | | | |
| 2D surface | | | |

As shown by the above table, the three-dimensional quality of the meshes provides increased surface area for cell attachment and proliferation compared to a planar 2D surface of comparable size. This increased surface area aids in the scalable performance achieved by embodiments of this disclosure. For process development and process validation studies, small-scale bioreactors are often required to save on reagent cost and increase experimental throughput. Embodiments of this disclosure are applicable to such small-scale studies, but can be scaled-up to industrial or production scale, as well. For example, if 100 layers of Mesh C in the form of 2.2 cm diameter circles are packed into a cylindrical packed bed with a 2.2 cm internal diameter, the total surface area available for cells to attach and proliferate is equal to about 935 cm². To scale such bioreactor ten times, one could use a similar setup of a cylindrical packed bed with 7 cm internal diameter and 100 layers of the same mesh. In such a case, the total surface area would be equal 9,350 cm². In some embodiments, the available surface area is about 99,000 cm²/L or more. Because of the plug-type perfusion flow in a packed bed, the same flow rate expressed in ml/min/cm² of cross-sectioned packed bed surface area can be used in smaller-scale and larger-scale versions of the bioreactor. A larger surface area allows for higher seeding density and higher cell growth density. According to one or more embodiments, the cell culture substrate described herein has demonstrated cell seeding densities of up to 22,000 cells/cm² or more. For reference, the Corning HyperFlask® has a seeding density on the order of 20,000 cells/cm² on a two-dimensional surface. As used herein, “plug-type perfusion flow” or “plug flow” refers to laminar flow through the bioreactor having a fixed bed according to embodiments herein, where the flow through any cross-section of the fixed bed perpendicular to the flow direction proceeds at the same rate across the cross section.

Another advantage of the higher surface areas and high cell seeding or growing densities is that the cost of the embodiments disclosed herein can be the same or less than competing solution. Specifically, the cost per cellular product (e.g., per cell or per viral genome) can be equal to or less than other packed bed bioreactors.

By using a structurally defined culture matrix of sufficient rigidity, high-flow-resistance uniformity across the matrix or packed bed is achieved. According to various embodiments, the matrix can be deployed in monolayer or multilayer formats. This flexibility eliminates diffusional limitations and provides uniform delivery of nutrients and oxygen to cells attached to the matrix. In addition, the open matrix lacks any cell entrapment regions in the packed bed configuration, allowing for complete cell harvest with high viability at the end of culturing. The matrix also delivers packaging uniformity for the packed bed, and enables direct scalability from process development units to large-scale industrial bioprocessing unit. The ability to directly harvest cells from the packed bed eliminates the need of resuspending a matrix in a stirred or mechanically shaken vessel, which would add complexity and can inflict harmful shear stresses on the cells. Further, the high packing density of the cell culture matrix yields high bioprocess productivity in volumes manageable at the industrial scale.

The geometry of the mesh substrate layers is designed to allow efficient and uniform flow through one or multiple substrate layers. In addition, the structure of the matrix can accommodate fluid flow through the matrix in multiple orientations. For example, the direction of bulk fluid flow can be perpendicular to the major side surfaces of the first and second substrate layers. However, the matrix can also be oriented with respect to the flow such that the sides of the substrate layers are parallel to the bulk flow direction. In addition to fluid flow being perpendicular or parallel to the first and second sides of the mesh layers, the matrix can be arranged with multiple pieces of substrate at intermediate angles, or even in random arrangements with respect to fluid flow. This flexibility in orientation is enabled by the essentially isotropic flow behavior of the woven substrate. In contrast, substrates for adherent cells in existing bioreactors do not exhibit this behavior and instead their packed beds tend to create preferential flow channels and have substrate materials with anisotropic permeability. The flexibility of the matrix of the current disclosure allows for its use in various applications and bioreactor or container designs while enabling better and more uniform permeability throughout the bioreactor vessel.

As discussed herein, the cell culture substrate can be used within a bioreactor vessel, according to one or more embodiments. For example, the substrate can be used in a packed bed bioreactor configuration, or in other configurations within a three-dimensional culture chamber. However, embodiments are not limited to a three-dimensional culture space, and it is contemplated that the substrate can be used in what may be considered a two-dimensional culture surface configuration, where the one or more layers of the substrate lay flat, such as within a flat-bottomed culture dish, to provide a culture substrate for cells. Due to contamination concerns, the vessel can be a single-use vessel that can be disposed of after use.

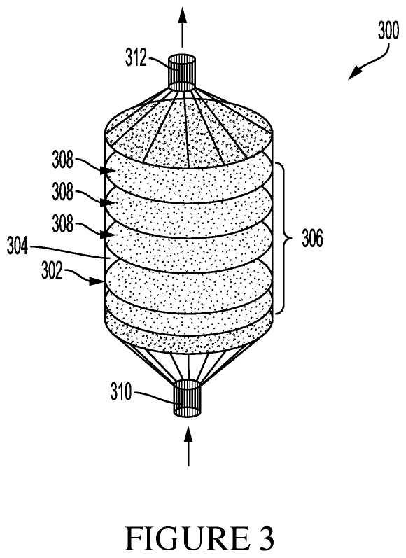

A cell culture system is provided, according to one or more embodiments, in which the cell culture matrix is used within a culture chamber of a bioreactor vessel. FIG. 3 shows an example of a cell culture system 300 that includes a bioreactor vessel 302 having a cell culture chamber 304 in the interior of the bioreactor vessel 302. Within the cell culture chamber 304 is a cell culture matrix 306 that is made from a stack of substrate layers 308. The substrate layers 308 are stacked with the first or second side of a substrate layer facing a first or second side of an adjacent substrate layer. The bioreactor vessel 300 has an inlet 310 at one end for the input of media, cells, and/or nutrients into the culture chamber 304, and an outlet 312 at the opposite end for removing media, cells, or cell products from the culture chamber 304. By allowing stacking of substrate layers in this way, the system can be easily scaled up without negative impacts on cell attachment and proliferation, due to the defined structure and efficient fluid flow through the stacked substrates. While the vessel 300 may generally be described as having an inlet 310 and an outlet 312, some embodiments may use one or both of the inlet 310 and outlet 312 for flowing media, cells, or other contents both into and out of the culture chamber 304. For example, inlet 310 may be used for flowing media or cells into the culture chamber 304 during cell seeding, perfusion, or culturing phases, but may also be used for removing one or more of media, cells, or cell products through the inlet 310 in a harvesting phase. Thus, the terms “inlet” and “outlet” are not intended to restrict the function of those openings.

In one or more embodiments, flow resistance and volumetric density of the packed bed can be controlled by interleaving substrate layers of different geometries. In particular, mesh size and geometry (e.g., fiber diameter, opening diameter, and/or opening geometry) define the fluid flow resistance in packed bed format. By interlaying meshes of different sizes and geometries, flow resistance can be controlled or varied in one or more specific portions of the bioreactor. This will enable better uniformity of liquid perfusion in the packed bed. For example, 10 layers of Mesh A (Table 1) followed by 10 layers of Mesh B (Table 1) and followed by 10 layers of Mesh C (Table 1) can be stacked to achieve a desired packed bed characteristic. As another example, the packed bed may start with 10 layers of Mesh B, followed by 50 layers of Mesh C, followed by 10 layers of Mesh B. Such repetition pattern may continue until the full bioreactor is packed with mesh. These are examples only, and used for illustrative purposes without intending to be limiting on the possible combinations. Indeed, various combinations of meshes of different sizes are possible to obtain different profiles of volumetric density of cells growth surface and flow resistance. For example, a packed bed column with zones of varying volumetric cells densities (e.g., a series of zones creating a pattern of low/high/low/high, etc. densities) can be assembled by interleaving meshes of different sizes.

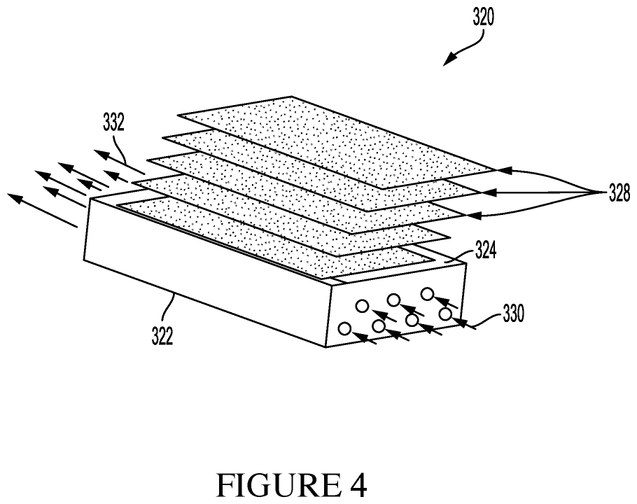

In FIG. 3, the bulk flow direction is in a direction from the inlet 310 to the outlet 312, and, in this example, the first and second major sides of the substrate layers 308 are perpendicular to the bulk flow direction. In contrast, the example shown in FIG. 4 is of an embodiment in which the system 320 includes a bioreactor vessel 322 and stack of substrates 328 within the culture space 324 that have first and second sides that are parallel to a bulk flow direction, which corresponds to a direction shown by the flow lines into the inlets 330 and out of the outlets 332. Thus, the matrices of embodiments of this disclosure can be employed in either configuration. In each of systems 300 and 320, the substrates 308, 328 are sized and shaped to fill the interior space defined by the culture chamber 304, 324 so that the culture spaces in each vessel are filled for cell growth surfaces to maximize efficiency in terms of cells per unit volume. Although FIG. 4 shows multiple inlets 330 and multiple outlets 332, it is contemplated that the system 320 may be fed by a single inlet and have a single outlet. However, according to various embodiments herein, distribution plates can be used to help distribute the media, cells, or nutrients across a cross-section of the packed bed and thus improve uniformity of fluid flow through the packed bed. As such, the multiple inlets 330 represent how a distribution plate can be provided with a plurality of holes across the packed-bed cross-section for creating more uniform flow.

The cell culture matrix can be arranged in multiple configurations within the culture chamber depending on the desired system. For example, in one or more embodiments, the system includes one or more layers of the substrate with a width extending across the width of a defined cell culture space in the culture chamber. Multiple layers of the substrate may be stacked in this way to a predetermined height. As discussed above, the substrate layers may be arranged such that the first and second sides of one or more layers are perpendicular to a bulk flow direction of culture media through the defined culture space within the culture chamber, or the first and second sides of one or more layers may be parallel to the bulk flow direction. In one or more embodiments, the cell culture matrix includes one or more substrate layers at a first orientation with respect to the bulk flow, and one or more other layers at a second orientation that is different from the first orientation. For example, various layers may have first and second sides that are parallel or perpendicular to the bulk flow direction, or at some angle in between.

In one or more embodiments, the cell culture system includes a plurality of discrete pieces of the cell culture substrate in a packed bed configuration, where the length and or width of the pieces of substrate are small relative to the culture chamber. As used herein, the pieces of substrate are considered to have a length and/or width that is small relative to the culture chamber when the length and/or width of the piece of substrate is about 50% or less of the length and/or width of the culture space. Thus, the cell culture system may include a plurality of pieces of substrate packed into the culture space in a desired arrangement. The arrangement of substrate pieces may be random or semi-random, or may have a predetermined order or alignment, such as the pieces being oriented in a substantially similar orientation (e.g., horizontal, vertical, or at an angle between 0° and 90° relative to the bulk flow direction).

The “defined culture space,” as used herein, refers to a space within the culture chamber occupied by the cell culture matrix and in which cell seeding and/or culturing is to occur. The defined culture space can fill approximately the entirety of the culture chamber, or may occupy a portion of the space within the culture chamber. As used herein, the “bulk flow direction” is defined as a direction of bulk mass flow of fluid or culture media through or over the cell culture matrix during the culturing of cells, and/or during the inflow or outflow of culture media to the culture chamber.

In one or more embodiments, the cell culture matrix is secured within the culture chamber by a fixing mechanism. The fixing mechanism may secure a portion of the cell culture matrix to a wall of the culture chamber that surrounds the matrix, or to a chamber wall at one end of the culture chamber. In some embodiments, the fixing mechanism adheres a portion of the cell culture matrix to a member running through the culture chamber, such as member running parallel to the longitudinal axis of the culture chamber, or to a member running perpendicular to the longitudinal axis. However, in one or more other embodiments, the cell culture matrix may be contained within the culture chamber without being fixedly attached to the wall of the chamber or bioreactor vessel. For example, the matrix may be contained by the boundaries of the culture chamber or other structural members within the chamber such that the matrix is held within a predetermined area of the bioreactor vessel without the matrix being fixedly secured to those boundaries or structural members.

One aspect of some embodiments provides a bioreactor vessel in a roller bottle configuration. The culture chamber is capable of containing a cell culture matrix and substrate according to one or more of the embodiments described in this disclosure. In the roller bottle configuration, the bioreactor vessel may be operably attached to a means for moving the bioreactor vessel about a central longitudinal axis of the vessel. For example, the bioreactor vessel may be rotated about the central longitudinal axis. The rotation may be continuous (e.g., continuing in one direction) or discontinuous (e.g., an intermittent rotation in a single direction or alternating directions, or oscillating in back and forth rotational directions). In operation, the rotation of the bioreactor vessel causes movement of cells and/or fluid within the chamber. This movement can be considered relative with respect to the walls of the chamber. For example, as the bioreactor vessel rotates about its central longitudinal axis, gravity may cause the fluid, culture media, and/or unadhered cells to remain toward a lower portion of the chamber. However, in one or more embodiments, the cell culture matrix is essentially fixed with respect to the vessel, and thus rotates with the vessel. In one or more other embodiments, the cell culture matrix can be unattached and free to move to a desired degree relative to the vessel as the vessel rotates. The cells may adhere to the cell culture matrix, while the movement of the vessel allows the cells to receive exposure to both the cell culture media or liquid, and to oxygen or other gases within the culture chamber.

By using a cell culture matrix according to embodiments of this disclosure, such as a matrix including a woven or mesh substrate, the roller bottle vessel is provided with an increased surface area available for adherent cells to attach, proliferate, and function. In particular, using a substrate of a woven mesh of monofilament polymer material within the roller bottle, the surface area may increase by of about 2.4 to about 4.8 times, or to about 10 times that of a standard roller bottle. As discussed herein, each monofilament strand of the mesh substrate is capable of presenting itself as 2D surface for adherent cells to attach. In addition, multiple layers of mesh can we arranged in roller bottle, resulting in increases of total available surface area ranging from about 2 to 20 times that of a standard roller bottle. Thus, existing roller bottle facilities and processing, including cell seeding, media exchange, and cell harvesting, can be modified by the addition of the improved cell culture matrix disclosed herein, with minimal impact on existing operation infrastructure and processing steps.

The bioreactor vessel optionally includes one or more outlets capable of being attached to inlet and/or outlet means. Through the one or more outlets, liquid, media, or cells can be supplied to or removed from the chamber. A single port in the vessel may act as both the inlet and outlet, or multiple ports may be provided for dedicated inlets and outlets.

The packed bed cell culture matrix of one or more embodiments can consist of the woven cell culture mesh substrate without any other form of cell culture substrate disposed in or interspersed with the cell culture matrix. That is, the woven cell culture mesh substrate of embodiments of this disclosure are effective cell culture substrates without requiring the type of irregular, non-woven substrates used in existing solution. This enables cell culture systems of simplified design and construction, while providing a high-density cell culture substrate with the other advantages discussed herein related to flow uniformity, harvestability, etc.

As discussed herein, the cell culture substrates and bioreactor systems provided offer numerous advantages. For example, the embodiments of this disclosure can support the production of any of a number of viral vectors, such as AAV (all serotypes) and lentivirus, and can be applied toward in vivo and ex vivo gene therapy applications. The uniform cell seeding and distribution maximizes viral vector yield per vessel, and the designs enable harvesting of viable cells, which can be useful for seed trains consisting of multiple expansion periods using the same platform. In addition, the embodiments herein are scalable from process development scale to production scale, which ultimately saves development time and cost. The methods and systems disclosed herein also allow for automation and control of the cell culture process to maximize vector yield and improve reproducibility. Finally, the number of vessels needed to reach production-level scales of viral vectors (e.g., 10¹⁶ to 10¹⁸ AAV VG per batch) can be greatly reduced compared to other cell culture solutions.

Embodiments are not limited to the vessel rotation about a central longitudinal axis. For example, the vessel may rotate about an axis that is not centrally located with respect to the vessel. In addition, the axis of rotation may be a horizonal or vertical axis.

### EXAMPLES

To demonstrate the efficacy of the cell culture matrix, cell culture systems, and related methods of this disclosure, studies were conducted on the seeding and culturing of cells, according to the following examples.

### Example 1

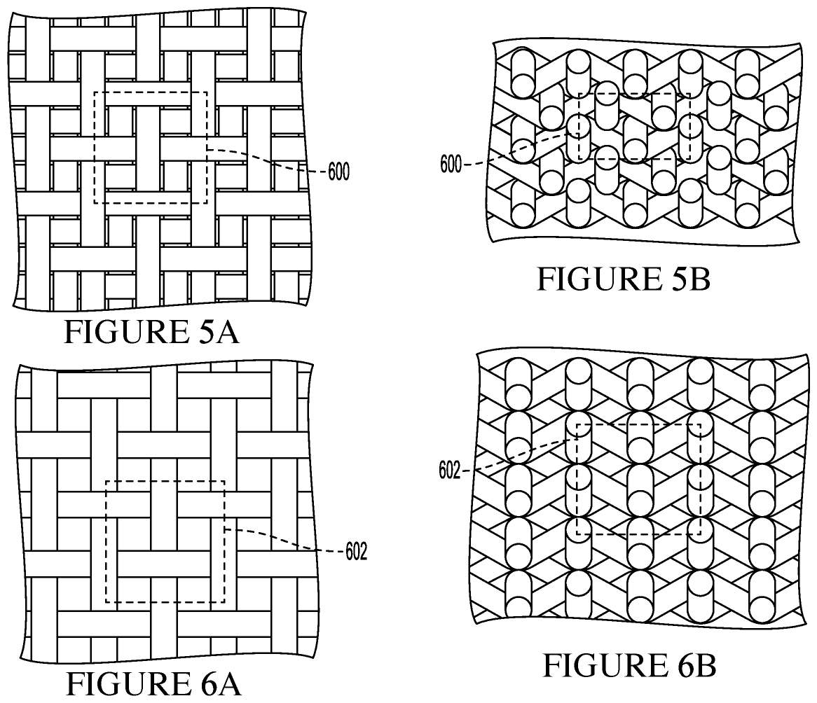

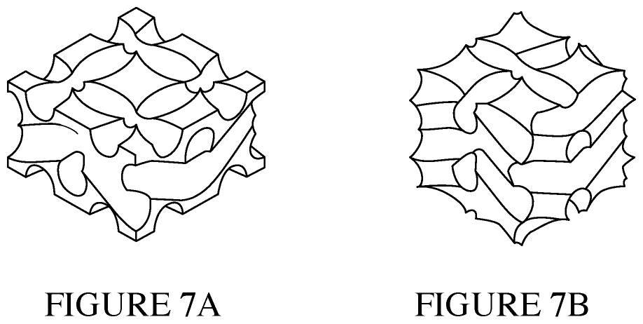

To further examine the flow uniformity or permeability of the substrates of this disclosure, modeling was used to understand the porosity of the three-dimensional cell culture matrix. Sheets of woven PET mesh substrate were modeled in a tight-packed configuration and a loose-packed configuration, which represent upper and lower boundaries of the packing density of a substrate stack for the particular mesh sheet that was modeling. In particular, FIG. 5A shows a plan view of the tight-packed configuration, and FIG. 5B shows a cross-section side view of the same stack. FIGS. 6A and 6B show plan and cross-section views, respectively, of the loose-packed configuration. For each modeled configuration, a sample cell 600, 602 was defined that encloses the same volume of mesh material to analyze the porosity per unit volume of the sample cell 600, 602. The modeled volume of open space within each cell is shown in FIGS. 7A (for tight-packed stack) and 7B (for loose-packed stack). The porosity in terms of percentage of open space was about 40.8% for the loose-packed cell, and 61.4% for the tight-packed cell. Because the modeled stacks in FIGS. 5A-6B represent the tightest-and loosest-packed configurations for the given mesh material, the porosities of 40.8% and 61.4% are the upper and lower bounds of porosity for this particular mesh material. Depending on the alignment and real-world packing density when using this mesh material, the porosity may fall in between these extremes. However, embodiments of this disclosure are not limited to this porosity range, as variations in the mesh dimensions and arrangement of the substrate within the cell culture vessel can lead to a different range of porosities.

In addition to the modeled porosity range, porosity was measured using real packed beds of PET woven mesh substrate. The measurements were made using one hundred disks, each with a diameter of 22.4 mm, stacked with random alignment. The total weight of the 100-disk stack was 5.65±0.2 g. Volume of the PET material of the stack was calculated, assuming a PET density of 1.38 g/cm³, using the following formula:

$${V}_{PET} = ( total weight of stack ) / ( density of PET ) \qquad Equation 2$$

Thus, the PET volume V_{PET} of 5.65 g of PET (for 100 disks of 22.4 mm diameter) was calculated to be 4.1 ml. The total volume Vₜₒₜₐₗ of the stack, including the PET volume V_{PET} and the volume of the open space within the stack, was then calculated using the following formula:

$${V}_{t otal} = π \times ( 0.5 \times disk diameter ) \times ( stacked bed height ) \qquad Equation 3$$

The 100-disk stack had a stack height of 25±1 mm. Thus, with a disk diameter of 22.4 mm, Vₜₒₜₐₗ was found to be 9.85 ml. Accordingly, porosity of the stacked bed can be calculated using the following:

$$Porosity = ( {V}_{total} - {V}_{PET} ) / {V}_{total} \qquad Equation 4$$

Using Equation 4 and the above values, the porosity was calculated to be 58.4%, which is within the range predicted by the model.

### Example 2

In Example 2, the permeabilities of various PET woven mesh substrate materials were compared. Table 5 shows the PET mesh samples used in this comparison.

TABLE 5
Mesh substrates for permeability comparison.

| | | Opening | Fiber | | Packing | Surface Area | Normalized |
|---|---|---|---|---|---|---|---|
| Mesh | Weave | Diameter | Diameter | Open | Thickness | of 60 mm | Surface to |
| Sample | Pattern | (μm) | (μm) | area | (μm) | disc (cm²) | Volume ratio |
| A | Plain | 250 | 160 | 37% | 280 | 74.9 | 1.00 |
| B | Twill | 250 | 152 | 39% | 280 | 74.2 | 0.99 |
| C | Plain | 210 | 147 | 35% | 230 | 80.9 | 1.16 |
| D | Plain | 200 | 112 | 41% | 130 | 68.1 | 1.30 |
| E | Plain | 300 | 195 | 37% | 370 | 68.1 | 0.83 |
| F | Plain | 319 | 128 | 51%* | 200 | 53.0 | 0.99 |

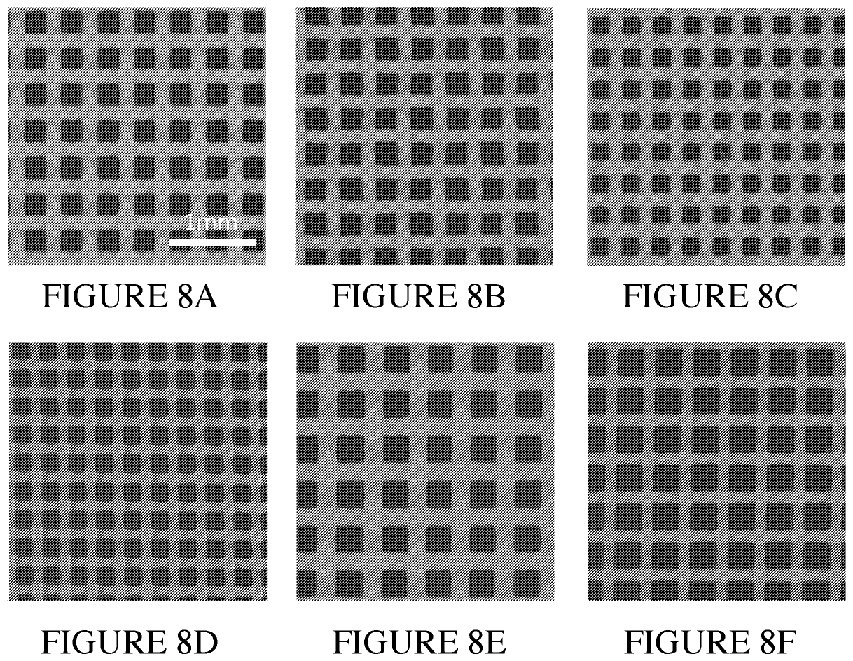

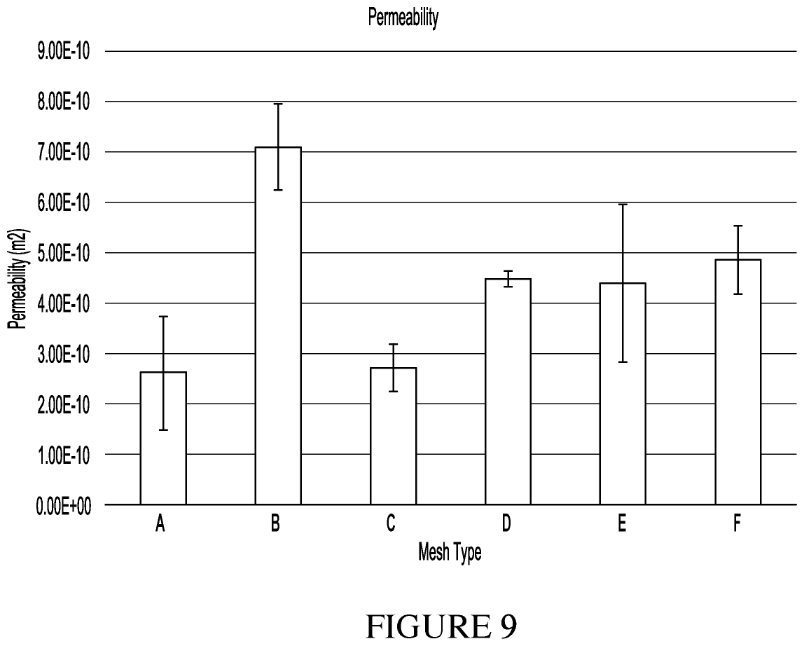

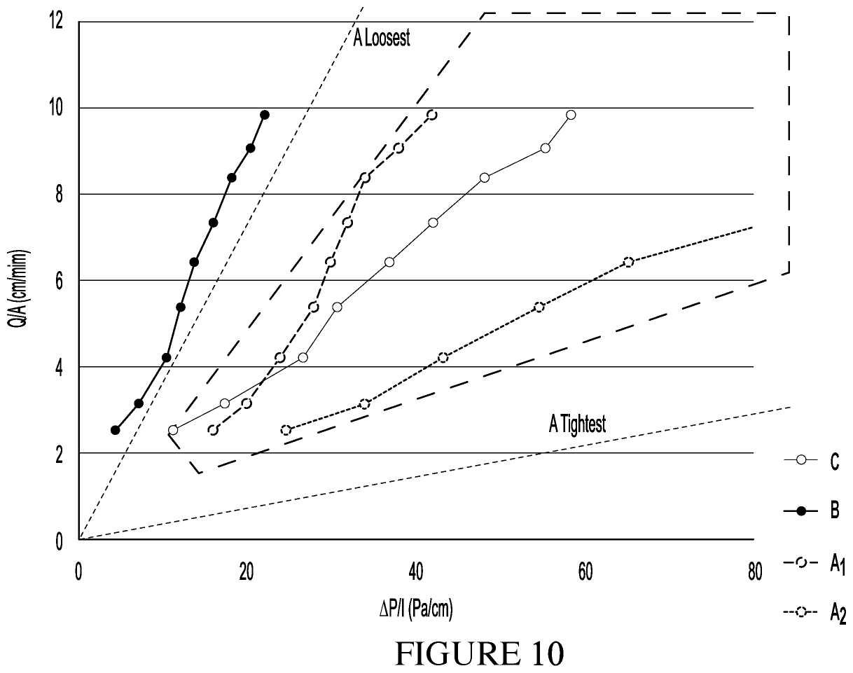

Photographs of the mesh samples A through F are shown in FIGS. 8A-8F. The results of the permeability of each mesh sample A-F is shown in FIG. 9. FIG. 10 shows the results of a pressure drop test for samples A-C, where a pressure drop test was conducted for sample A in stacks with different arrangements and packing densities The dotted lines represent the tightest and loosest packing densities for mesh sample A, with sample A₁ being a more loosely packed stack than sample A₂. The pressure drop in terms of change in pressure (Pa) per centimeter is plotted against Q/A.

### Example 3

As discussed herein, the embodiments of this disclosure provide cell culture substrates, bioreactor systems, and methods of culturing cells or cell by-products that are scalable and can be used to provide a cell seed train to gradually increase a cell population. One problem in existing cell culture solutions is the inability for a given bioreactor system technology to be part of a seed train. Instead, cell populations are usually scaled up on various cell culture substrates. This can negatively impact the cell population, as it is believed that cells become acclimated to certain surfaces and being transferred to a different type of surface can lead to inefficiencies. Thus, it would be desirable to minimize such transitions between cell culture substrates or technologies. By using the same cell culture substrate across the seed train, as enabled by embodiments of this disclosure, efficiency of scaling up a cell population is increased. For example, the seed train can begin with a vial of starter cells which are seeded into a first vessel (such as a T175 flask from Corning), then into a second vessel (such as a HyperFlask® from Corning), then into a process-development scale bioreactor system according to embodiments of this invention (effective surface area of substrate of about 20,000 cm²), and then into a larger bioreactor pilot system according to embodiments of this invention (effective surface area of substrate is about 300,000 cm²). At the end of this seed train, the cells can be seeded into a production-scale bioreactor vessel according to embodiments of this disclosure, with a surface area o about 5,000,000 cm², for example. Harvest and purification steps can then be performed when the cell culture is complete. Harvest can be accomplished via in situ cell lysis with a detergent (such as Triton X-100), or via mechanical lysis; and further downstream processing can be performed, as needed.

The benefits of using the same cell culture substrate within the seed train (e.g., from process development level to pilot level, or even to production level) include efficiencies gained from the cells being accustomed to the same surface during the seed train and production stages; a reduced number of manual, open manipulations during seed train phases; more efficient use of the packed bed due to uniform cell distribution and fluid flow, as described herein; and the flexibility of using mechanical or chemical lysis during viral vector harvest.

### Example 4

To understand the potential increased viral production yield of substrates of the present disclosure, the performance of a PET woven mesh substrate was compared to that of a substrate material similar to the substrate used in the iCellis®. Table 6 summarizes the total viral particles produces using these substrates in a simplified bioreactor.

TABLE 6
Viral particles produced using PET woven mesh
substrate material and non-woven substrate.

| Test | PET Woven | Non-woven |
|---|---|---|
| No. | Mesh | Substrate |
| 1 | 6.99E13 | 7.18E12 |
| 2 | 3.65E13 | e |
| 3 | 7.72E13 | 1.01E13 |
| 4 | 4.63E13 | n/a |
| Average | 6.01E13 | 1.01E13 |

From the results in Table 6, it is possible to calculate the volume of substrate material needed to produce a certain number of viral particles. For example, if the goal for production-scale viral vector production is 3.00E+18 viral particles, as shown in Table 7, the volume of PET woven mesh needed is about one-seventh the amount of the non-woven substrate needed.

TABLE 7
Comparison of PET woven mesh substrate and non-woven substrate

| | | | | ml of substrate | M3 of substrate |
|---|---|---|---|---|---|
| | Total | Reactor bed | Crude VP | needed to produce | material needed to |
| Substrate | VP/reactor | volume | needed | crude 3E18 VP | produce crude 3E18 VP |
| PET mesh | 6.01E+13 | 15 | 3.00E+18 | 7.49E+05 | 0.7 |
| Non-woven | 1.01E+13 | 15 | 3.00E+18 | 4.46E+06 | 4.5 |
| substrate | | | | | |

### Example 5

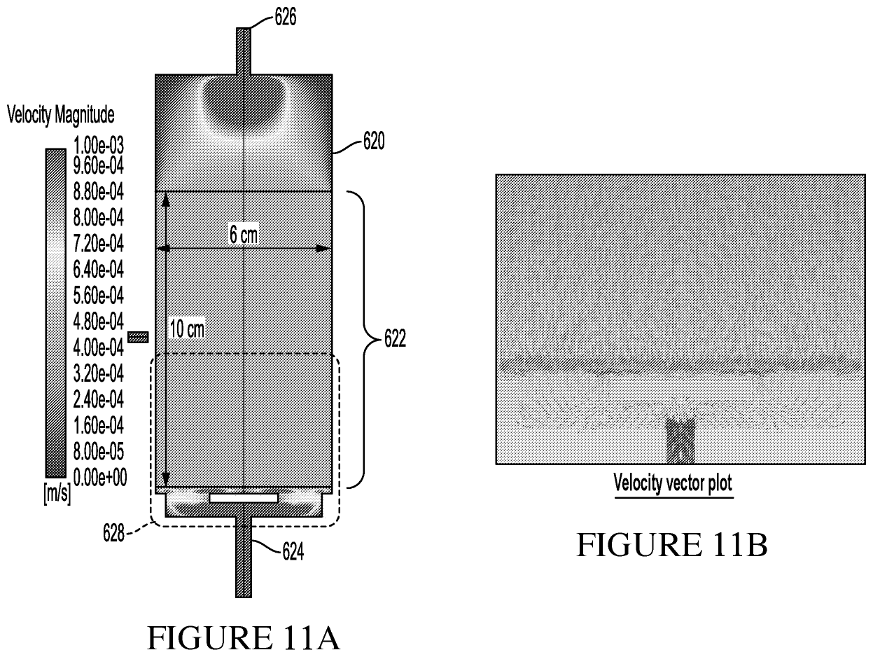

To demonstrate the uniform flow through the open mesh substrates of this disclosure, fluid flow velocity through a packed bed bioreactor was modeled. FIG. 11A shows the modeling results for a vessel 620 having a packed bed region 622 of PET woven mesh disks with diameters of 6 cm, and a bed height of 10 cm and consisting of 357 disks of PET woven mesh substrate. Fluid velocity magnitude is depicted according to the scale shown. While the flow velocity is high near the inlet 624 and outlet 626, the velocity is constant throughout the packed bed region 622, including along the height and across the width of the packed bed. The region denoted by the dotted line 628 is shown in close-up in FIG. 11B, which shows that once the fluid enters the uniform, open structure of the cell culture matrix, velocity is relatively constant. The packed bed in this example equates to a total surface of about 24,214 cm². Given the uniform flow shown in the model, the percentage of this surface area exposed to non-uniform flow (defined as >2.5% deviation from the mean velocity) was 0%.

To demonstrate the extent to which this uniform flow persists as the vessel is scaled up in size, additional modeling was conducted similar to that of FIG. 11A, but using progressively wider vessels with wider packed beds. The percentage of non-uniform flow for these larger vessel is shown in Table 8. As shown, even when the reactor is scaled up to a diameter of 60 cm, the amount of non-uniform flow remains about one-half of one percent or less of the surface area of the substrate. This shows that the uniform, open woven mesh structure described herein is capable of uniform flow throughout the entirety of the packed bed, unlike existing cell culture substrates.

TABLE 8
Modeled flow uniformity for bioreactors
having packed beds of various diameters.

| Packed bed | Total SA | % of SA with non |
|---|---|---|
| diameter (cm) | (cm²) | uniform flow* |
| 6 (100 disks) | 6,780 | 0.00 |
| 6 (357 disks) | 24,214 | 0.00 |
| 20 | 269,047 | 0.00 |
| 30 | 605,357 | 0.17 |
| 40 | 1,076,190 | 0.52 |
| 60 | 2,421,428 | 0.45 |

### Example 6

To better understand the difference in permeability between the woven mesh substrate of the present disclosure and the non-woven, irregular substrates on the existing market, experiments were conducted to measure the permeability of these materials. In particular, a PET woven mesh was compared to a non-woven substrate material and to a similar non-woven, disordered substrate that was commercially available. Permeability measurements were made for flow perpendicular to the woven mesh substrate layers of a packed bed of stacked disks, and through a randomly packed bed of non-woven substrate, as well as through a fixed sheet of non-woven substrate material. The non-woven mesh had a fiber diameter of about 20 μm, a thickness of about 0.18 mm, and a porosity o 91%. The woven mesh substrate had a diameter of about 160 um and an opening diameter of 250 μm.

To measure permeability, water was used for the test to simulate cell culture medium. The flow rate was controlled to be between 15-50 ml/cm²/min using a peristaltic pump to simulate flow conditions that substrates typically experience in a bioreactor. Due to low pressure drop the samples experienced under the test condition, a monometer was used to measure the pressure difference across the samples. Because of the different substrate types and packing methods, the substrates where held in slightly different ways.

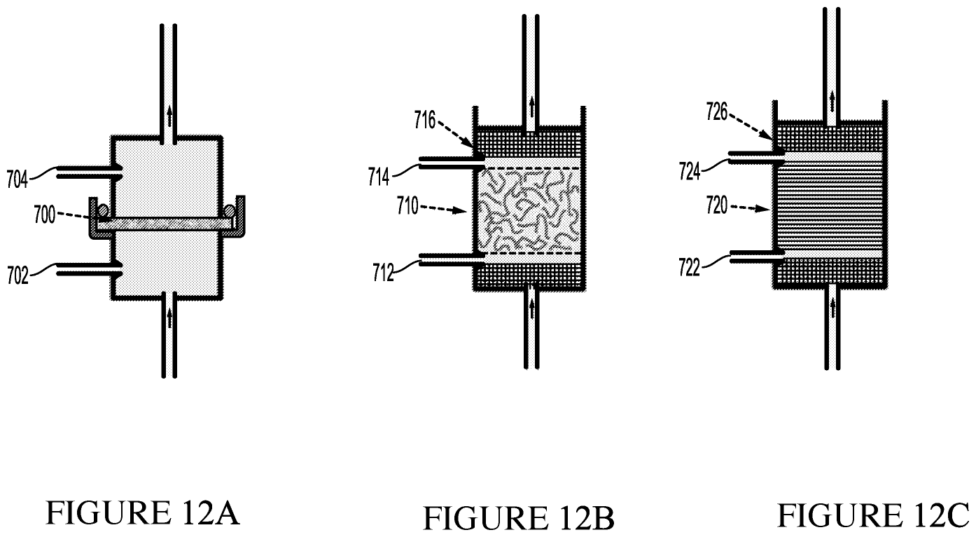

As shown in FIG. 12A, to measure flow across the non-woven mesh, the non-woven substrate 700 was cut into 12 mm diameter discs and ten levels of the substrate were held between two open cylindrical chambers sealed by an O-ring. Monometers 702, 704 were connected directly to the open chamber to measure the pressure drop of the fluid across the non-woven substrate.

To measure flow through randomly packed non-woven mesh, as shown in FIG. 12B, the substrate was cut into 5 mm×25 mm strips and packed into a 29 mm diameter cylindrical chamber creating a packed bed 710. A total of 3 g of mesh strips were packed into a 30 ml volume, resulting in a bed height of about 45 mm. At each side of the packed strips, two discs of open woven mesh was used to confine packed bed. There was about a 3 mm-thick open space at each side of the bed where two pieces of porous material 716 with thickness of 10 mm were used to redistribute the flow at the inlet and outlet of the vessel. Monometers 712 and 714 measured the pressure drop across the packed bed 710.

To measure flow through open woven mesh, as shown in FIG. 12C, the mesh substrate was cut into 29 mm-diameter discs to fit into the cylindrical chamber as a packed bed 720. A total of 170 discs were packed layer-by-layer until a bed height of 45 mm was achieved. The orientations of fibers in each level of mesh were not aligned to each other. The flow was across the mesh discs (i.e., perpendicular to the disk surfaces). There was about a 3 mm-thick open space at each size of the bed then two pieces of porous material 726 with thickness of 10 mm were used to redistribute the flow at inlet and outlet. Monometers 722 and 724 measured the pressure drop across the packed bed 720.

The permeability was calculated using Equation (5):

$$Q = - \frac{KA}{μ} \frac{dP}{d L} \qquad Equation ( 5 )$$

Where: Q=flow rate; K=permeability; A=across area of the sample or packed bed; dP=pressure drop across the test sample or packed bed; μ=water viscosity; and dL=total sample thickness or packed bed height

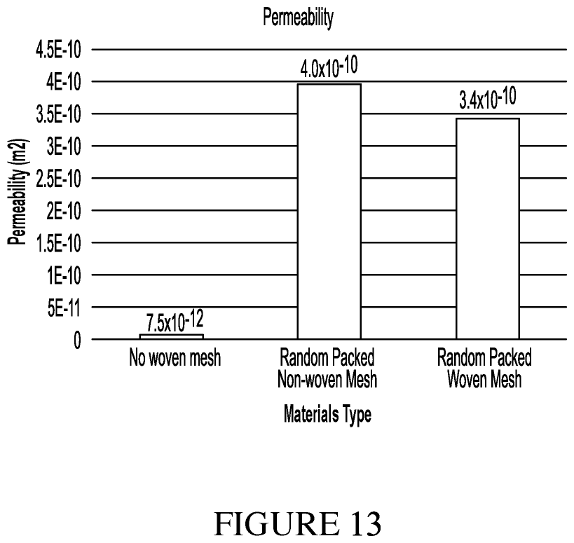

The final calculated permeabilities are summarized in FIG. 13. The results show that non-woven mesh had much lower permeability was of about 7.5×10⁻¹² m², which was about 1/50 of the permeability across the open woven mesh. When the non-woven mesh was cut into small strips and packed randomly, their permeability increased enormously and became similar as open woven mesh. This increased permeability is believed to be the result of the flow mostly bypassing around the mesh strips due to the channeling effect discussed above.

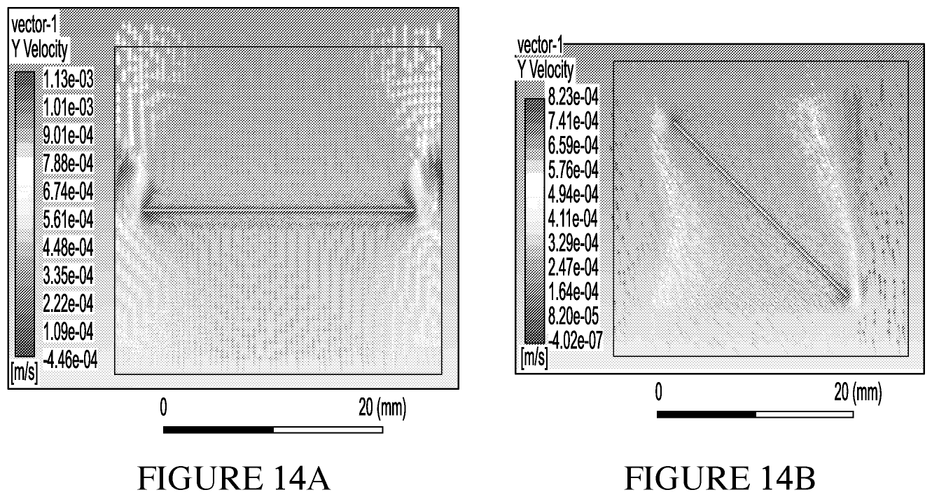

Based on the measured permeabilities, the flow was simulated through and around the non-woven mesh and the open woven mesh. The simulation was done using ANSYS Fluent v19.2 software package. For illustration purpose, two scenarios were studied: with the surface of the substrate material surface aligned (i) 90° and (ii) 45° with respect to the flow direction, as shown in FIGS. 14A and 14B. In both cases, the flow was mostly around the mesh and only about 0.02˜0.005% of the flow went through the mesh, when the spacing between neighboring mesh on the same level is 5 mm. This will create a significant dead zone behind the mesh and cause the non-uniform flow through the packed bed. The non-uniformity becomes more severe when the non-woven mesh piece is not perfectly aligned normal to the flow direction.

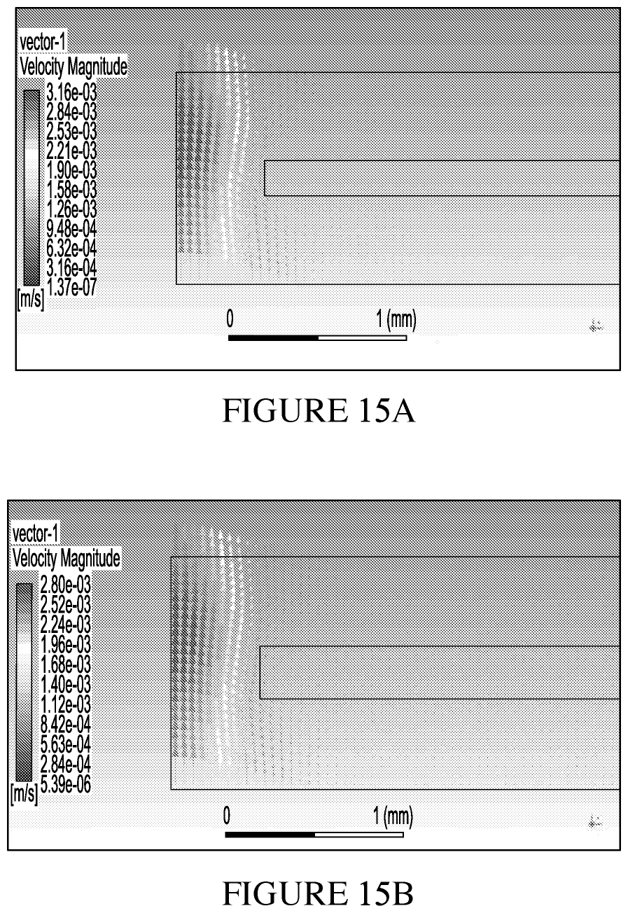

In the case of open woven mesh, the open structure made allowed flow easily through the mesh and did not create a dead zone behind the open mesh layer. It is believed that the regular structure of the woven mesh also contributed to the uniform flow distribution through each level. This, in turn, enables more uniform flow in through the entire packed bed. The comparison is clearly shown in FIGS. 15A (non-woven mesh piece) and 15B (open woven mesh substrate), which show a close-up view of flow near the edge of the substrate materials. In the case of FIGS. 15A and 15B, the gap between neighboring mesh pieces in all six directions is shortened to 1 mm, and only the periodic domain of one such mesh piece is simulated. FIG. 15A shows that with non-woven mesh has very low permeability, as the flow is mostly bypassing around the substrate, and there is very little flow through the substrate itself. Only 0.17% of the total mass flow goes through the non-woven mesh. In contrast, open woven mesh has a much higher permeability, so that there is more flow through it as shown in FIG. 15B. The shortcut flow through the gap becomes weaker when comparing the colorbars in the two cases. For the open woven mesh, there is as high as 10.7% of the total mass flow rate going through the substrate, which demonstrates that open woven mesh has superior permeability even if it is packed with gaps.

As discussed herein, it is possible for multiple woven mesh disks to be randomly packed with countless variations in alignment between the disks. However, the range of possible alignments can be reduced to two theorical limits based the packing density (i.e. the tightest and the loosest packing). These two ideal or boundary conditions allow for simplifying a large packed bed to a small periodic domain. Using this model, it was found that permeability through the substrate differs by roughly 10 times from the tightest to the loosest packing limit. The experimentally measured permeability data from above lies well within this range, which served as a good validation point. The model also showed that permeability in the flow direction is similar to that in the traverse direction, in both packing conditions. This suggests that the woven mesh of this disclosure will less likely change the flow direction as we found in non-woven substrates and make the flow more uniform, regardless of substrate orientation. The improved flow uniformity of substrates of this disclosure was further demonstrated by the residence time distribution (RTD) measurements in the following example.

### Example 7

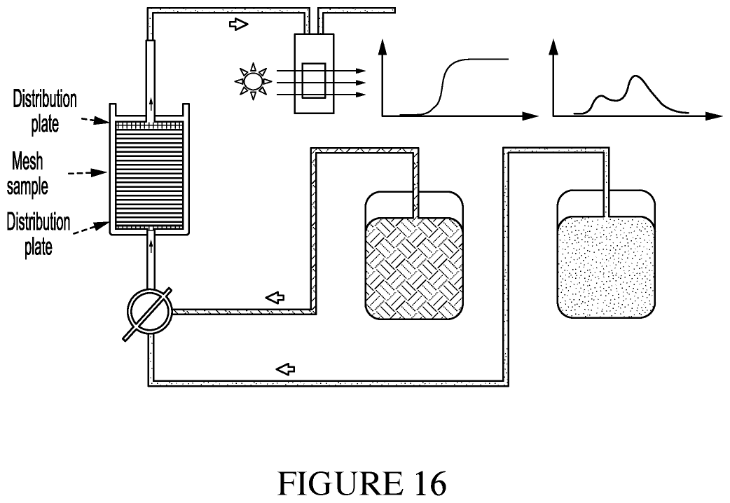

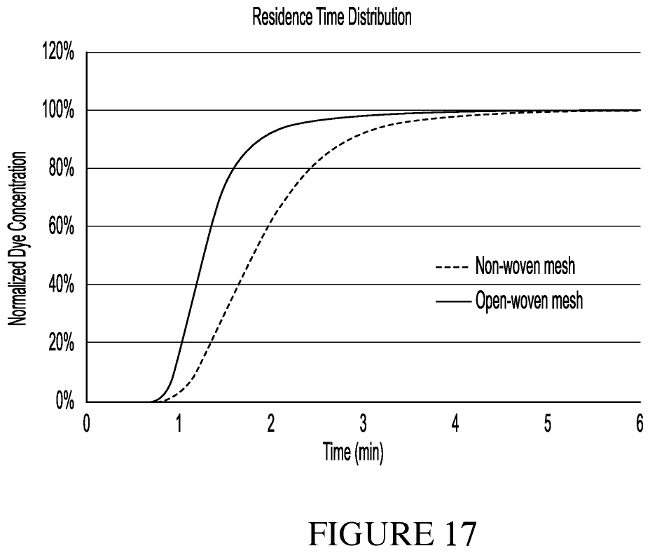

Residence time distribution (RTD) is a useful tool to study the flow in a vessel. Its theory, measurement, and analysis can be found in the textbook: Levenspiel, O. Chemical Reaction Engineering. 3ed. 1999. Wiley. New York. FIG. 16 shows the schematic drawing of the setup to measure RTD. The bioreactor vessel containing the fixed bed is cylindrical with diameter of 29 mm and the total fixed bed volume is 36 ml. The fixed bed consisted of 3.6 g of non-woven mesh or, according to embodiments of this disclosure, 200 layers of open woven mesh. 1:2000 diluted McCormick Green Food Color was used as a tracer for the RTD measurement. A UV-vis with a Flowcell was used to monitor the change of tracer concentration. A flow rate of 22.5 ml/ml was used for all experiment. The chamber was first filled with water. After switched to green dye, the change of the optical density (OD) was recorded. The results in terms of normalized dye concentration versus time are shown in FIG. 17. As shown in FIG. 17, the non-woven mesh resulted in slower increase in dye concentration over a longer period of time as compared to the open woven mesh. For example, the normalized dye concentration for the non-woven mesh has a lower slope than for the open woven mesh, and takes more time to reach a comparable normalized concentration level.

The following equations were used to calculate mean residence time t (Equation (6)) and variance σ (Equation (7)):

$$\overline{t} = {\int }_{0}^{∞} ( 1 - F ) dt \qquad Equation ( 6 )$$

$${σ}^{2} = 2 {\int }_{0}^{∞} t ( 1 - F ) d t - {\overline{t}}^{2} \qquad Equation ( 7 )$$

where F is normalized concentration in a step tracer response. Table 9 summarizes the calculated mean residence time and variance from the measurement. The open woven mesh shows shorter mean residence time, which was likely caused by the lower porosity and decreased dead zones. In a packed bed of open woven mesh, the porosity was about 60% while the porosity of the non-woven mesh has higher porosity which was about 93%. The much higher normalized variance detected in packed bed of non-woven mesh suggests that the flow in non-woven mesh was less uniform or ideal.

TABLE 9
Means residence time and variance of non-woven
and open woven mesh from measurement.

| | Woven mesh | Non-woven mesh |
|---|---|---|
| Mean residence time (min) | 1.37 | 1.97 |
| Variance (min²) | 0.21 | 0.84 |
| Normalized variance | 0.11 | 0.22 |

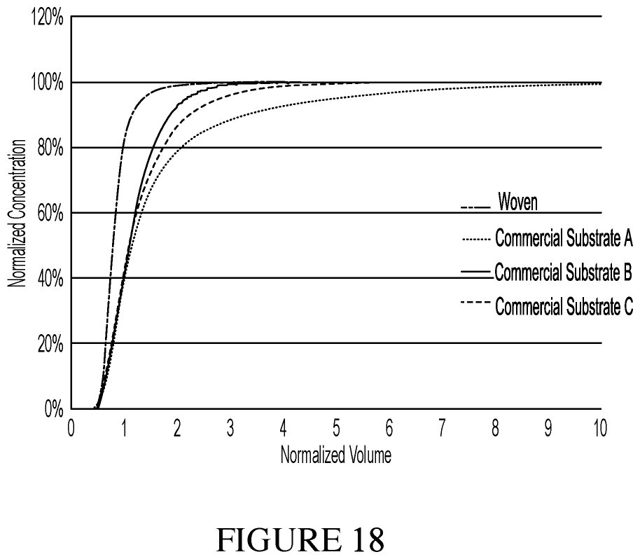

Using the same setup as described above, the RTDs of other commercially available packed bed substrate materials were measured. The packing density was calculated based on the vendor recommendation and to avoid loose packing. The change of dye concentration with the volume of water flowed through the vessels are shown in FIG. 18. The measured changes of dyes were used to calculate their normalized variance as described in previous section. The calculated variances are listed in Table 10.

TABLE 10
Normalized variance of different commercially available packed bed
substrates vs. a woven mesh substrate of the present disclosure.

| | |
|---|---|
| Packing density | Normalized variance |

| | | | |
|---|---|---|---|
| Woven mesh | 1.8 | ml/g | 0.11 |
| Commercial Substrate 1 | 7.5 | ml/g | 0.94 |
| Commercial Substrate 2 | 6 | ml/g | 0.24 |
| Commercial Substrate 3 | 10 | ml/g | 0.34 |

From the above permeability and residence time experiments, it is shown that the type of non-woven, irregular cell culture substrate used in current bioreactors has lower permeability than the substrate of the present disclosure. These non-woven substrates also have different permeability or flow rates depending on the direction of flow relative to the non-woven substrate, whereas the substrates of the present disclosure exhibit essentially isotropic flow behavior. Due to the non-uniform flow and lower residence time of the non-woven substrates, nutrients and transfection reagents can take longer to reach to the cells on the substrate surface or the other side of a substrate layer, as compared to the uniform, woven mesh substrate of the present disclosure. Adding to this is the higher permeability of the randomly packed non-woven substrate, which suggest a strong channeling effect and thus non-uniform delivery of cells or nutrients.

### Example 8

To further quantify the flow uniformity in substrates according to embodiments of this disclosure, the compaction or occupancy of the fixed bed was controlled in experiments using a setup similar to that shown in FIG. 17. Specifically, to compare and quantify the flow uniformity through different fixed beds, the RTD was measured. In each experiment, while maintaining flow through the bioreactor fixed bed, a small volume of a green dye solution was injected at the inlet of the bioreactor and the change of dye concentration over time was measured at the outlet at the outlet. The dye was injected over a direction of within 2 seconds, which is considered as a pulse input. The change in dye concentration at the outlet was recorded using a UV-vis spectrometer. Different flow rates were used during the measurement depending on the experimental designs.

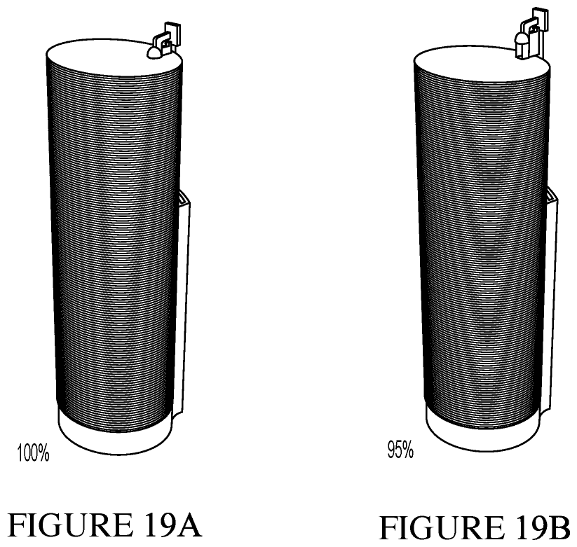

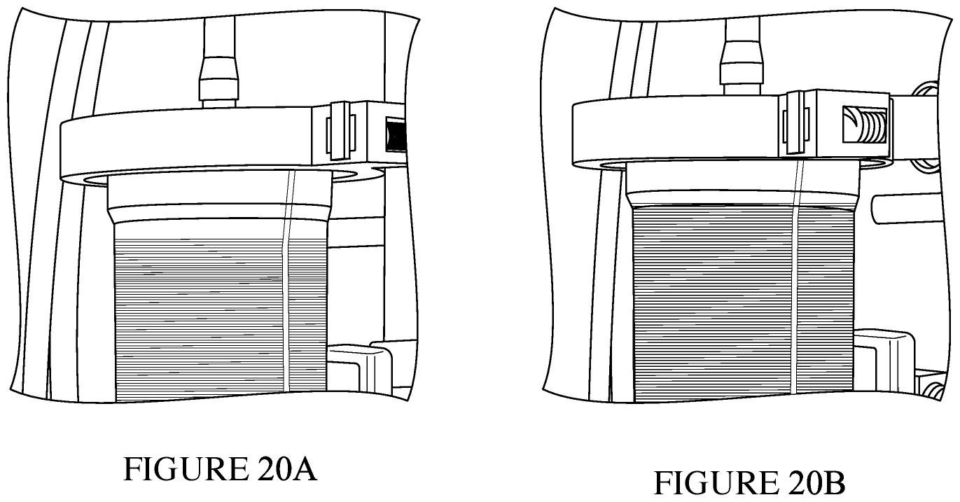

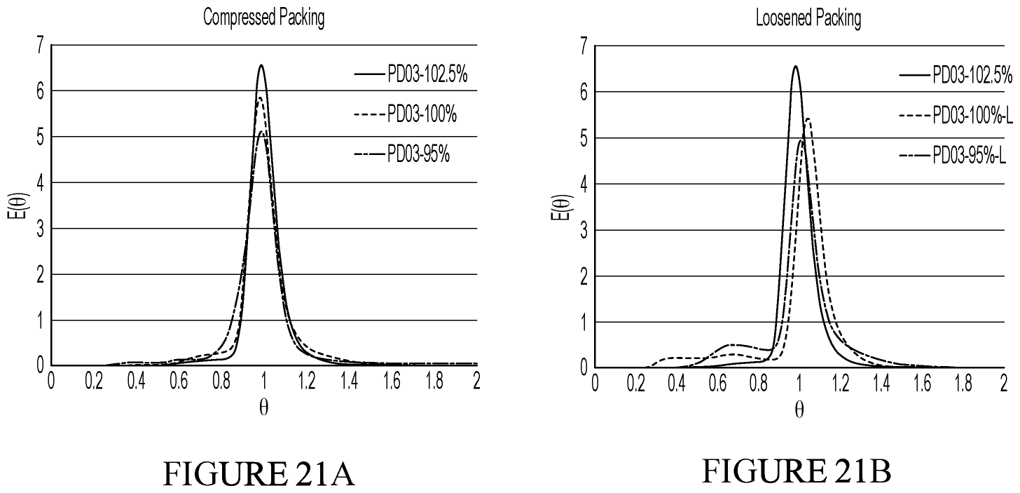

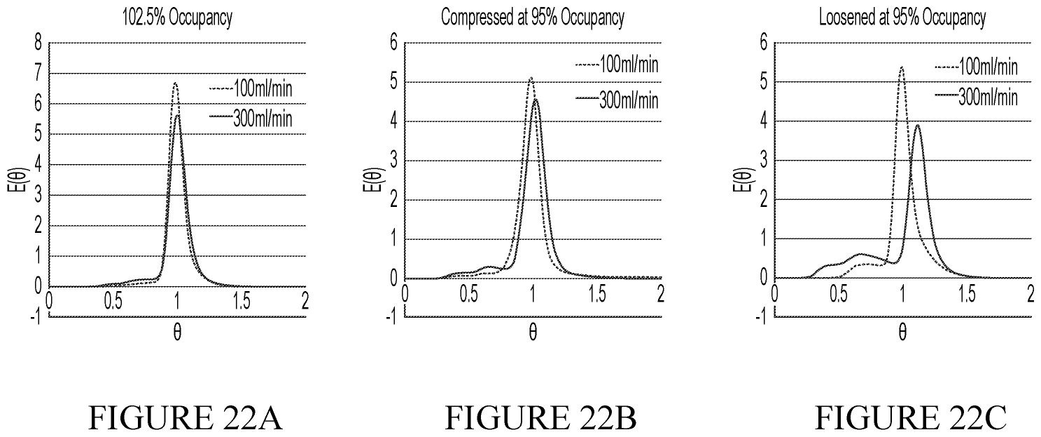

The reactor used in these experiments had an internal diameter of 63 mm and 201.7 mm bed space height to hold substrate material as a fixed bed or packed bed. Substrates included open woven mesh disks, according to embodiments of this disclosure. To pack the reactor, each disk was aligned relative to a disk according to a 45° rotation about a z-axis, where the z-axis is parallel to the longitudinal axis of the bioreactor bed space and is perpendicular to the first and second major surfaces of the disk. Thus, the fibers in each woven disk were rotated 45° relative to the fibers in the neighboring disk. This rotation prevented interlocking of the fibers between disks and provided more consistent permeability. To control the packing density or occupancy, we used the space height of the reactor divided by the thickness of each mesh disk to obtain the number of disks for 100% occupancy. In this situation, the mesh disks filled all the way to the top of reactor space. FIG. 19A shows this stack of disks at 100% occupancy. By removing 5% of the disk count from the 100% occupancy condition, we obtained 95% occupancy as shown in FIG. 19B.

At lower occupancy, either the disks were compressed down, in which case empty space was created above the packed bed (this is referred to as compressed packing), as shown in FIG. 25A, or the extra space was distributed across the packed bed without clear space on the top as shown in FIG. 25B (this is referred to as loose packing). Even at 100% occupancy, due to some elasticity of the mesh layer, the bed can still be compressed to some extent and result in a little space left above the fixed bed. This is called compressed packing at 100% occupancy, whereas without the applied compression there would be loose packing. In compressed configuration, about 2.5% extra disks were added to create 102.5% occupancy which means that the mesh disks were always maintained at highly compressed condition.

The experiments showed that compressed packing, including different occupancies from 95% to 102.5%, surprisingly provided good and comparable RTD profiles, demonstrating flow uniformity, as shown in FIG. 26A. However, loose packing generated comparably worse results, which indicated more bypass at 100 ml/min, as shown in FIG. 26B, as represented by a larger earlier peak of the E(θ) curve. With increased flow rate, such as 300 ml/min, the bypass was increased further, and more significant for lower occupancies and loose packing conditions, as demonstrated in FIG. 26B and FIGS. 27A-C. This means that, when a higher flow rate is needed during higher density cell culture, the flow uniformity will suffer more.

FIGS. 27A, 27B, and 27C show the E(θ) curves using substate disks according to embodiments of this disclosure under different packing conditions. In FIG. 27A, the bed is packed at 102.5% occupancy. In FIG. 27B, the bed is packed at 95% occupancy with compressed packing. In FIG. 27C, the bed is paced at 95% occupancy with loose packing. As shown, the loose packing condition in FIG. 27C results in an early peak (indicating channeling), a wider base of the E(θ) curve, and more variability between flow rates, with a higher flow rate creating worse flow. In comparison, compressed packing, at both 102.5% and 95% occupancy, creates more uniform flow conditions that do not vary significantly with flow rate.

It should be noted that the E(θ) curve is normalized and dimensionless, and thus bioreactors of different sizes can be easily compared on the same curve. According to embodiments of this disclosure, the substate and fixed beds show remarkably consistent E(θ) curves regardless of the size of the reactor. For example, a fixed bed having a substrate surface area of 5 m² and one having a surface area of 100 m² have similar overlapping E(θ) curves. These sizes are given for example, only, as it is anticipated that fixed beds of any size according to embodiments herein will have similar flow characteristics.

As would be understood by a person of ordinary skill in the art, an “early” peak means that the peak of the E(θ) comes earlier θ=1 (and, in the above experiment, earlier than those of the compressed packing). A distribution of the RTD curve closer to p74 =1 indicates more uniform flow or flow closer to a true “plug flow” condition. For example, a single, sharp peak in the E(θ) curve indicates that all (or nearly all) dye particles injected spend about the same amount of time in the reactor, which indicates uniform flow that is close to idealized “plug flow”. However, in some substrates or beds, channeling can occur meaning that the permeability of the bed will be variable and the particles of dye used in the RTD experiment will spend variable amounts of time in the reactor. This can be indicated by an “early” peak on the E(θ) curve, indicating dye that reached the bioreactor outlet early, and/or by a wider E(θ) curve, indicating a longer length of time to clear the dye from the reactor.

Without wishing to be bound by theory, it is believed that with loose packing, the substrate disks may not be able to keep flat in the fixed bed due to less restriction from neighboring disks. Lifted or curved disks can create more gaps between the disks and vessel wall, and lead to more bypass. At higher flow rate, the flow can further lift the disks and make greater gaps and more bypass flow.

### Example 9

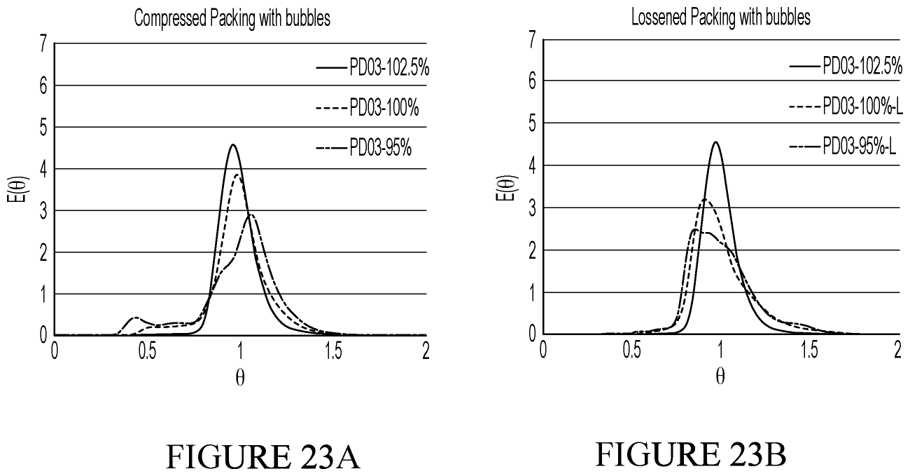

To simulate the effect of bubbles, the setup described above for Example 8 was used, but 8 ml of air was injected at the inlet before corresponding RTD measurements. The obtained RTD profiles were compared with the same reactor before bubble introduction. As shown in FIGS. 23A and 23B, at lower occupancies, the RTD profile, or E(θ) curve, has a wider spread, suggesting poor uniformity compared to those at higher occupancies. This was similar for both compressed packing (FIG. 23A) and loose packing (FIG. 23B).

Without wishing to be bound by theory, it is believed that, at high occupancy such as 100% or 102.5%, there is not much extra space in the packed bed for bubbles and most of the bubbles were pushed out of the bed by perfusion flow through the reactor, which made the impact much less. While at lower occupancies, bubbles were easily trapped in the extra spaces in the bed and then disturbed the perfusion flow through the bed. Since bubbles are difficult to avoid during cell culture, using high occupancy can help minimize the negative impact of bubbles, and maintain uniform flow to support better cell growth.

Embodiments of this disclosure includes aspects of embodiments disclosed in U.S. patents application Ser. Nos. 16/781,685; and 16/781,723; and U.S. Provisional Patent Application No. 63/227,693, the contents of which are hereby incorporated herein in their entireties.

### Illustrative Implementations

The following is a description of various aspects of implementations of the disclosed subject matter. Each aspect may include one or more of the various features, characteristics, or advantages of the disclosed subject matter. The implementations are intended to illustrate a few aspects of the disclosed subject matter and should not be considered a comprehensive or exhaustive description of all possible implementations.

Aspect 1 pertains to a bioreactor system for culturing cells, the system comprising: a cell culture vessel comprising at least one interior reservoir, an inlet fluidly connected to the reservoir, and an outlet fluidly connected to the reservoir; and a cell culture matrix disposed in the reservoir, the cell culture matrix comprising a substrate configured for adhering cells thereto, wherein the cell culture matrix is configured to maintain a uniform flow of liquid media through the cell culture matrix, such that the uniform flow satisfies the following expressions:

$${\int }_{θ 1}^{θ 2} E ( θ ) d θ = P ,$$

$$Δθ = {θ}_{2} - {θ}_{1} , and$$

$$Δθ \leq N ,$$

wherein P is greater than or equal to at least one of 0.3, 0.4, 0.5, 0.6, 0.7, 0.8, 0.9, and 0.95, and wherein N is less than or equal to at least one of 0.1, 0.2, 0.3, 0.4, 0.5, 0.6, 0.7, 0.8, 0.9, and 1.0.

Aspect 2 pertains to the bioreactor system of Aspect 1, wherein N is less than or equal to 0.5, and P is greater than or equal to 0.7.

Aspect 3 pertains to the bioreactor system of Aspect 1 or 2, wherein E(θ) is less than or equal to 0.2 or less than or equal to 0.1, when θ is less than or equal to 0.8, 0.75, 0.7, 0.6, or 0.5.

Aspect 4 pertains to the bioreactor system of any one of Aspects 1-3, wherein E(θ) is less than or equal to 0.2 or less than or equal to 0.1, when θ is greater than or equal to 1.2, 1.25, 1.3, 1.4, or 1.5.

Aspect 5 pertains to the bioreactor system of any one of Aspects 1-4, wherein E′(θ)=0 at a value of θ between 0.6 and 1.4; between 0.7 and 1.3; between 0.8 and 1.2; and between 0.9 and 1.1.

Aspect 6 pertains to the bioreactor system of any one of Aspects 1-5, wherein the cell culture matrix is configured to maintain the uniform flow of liquid media through the cell culture matrix with a normalized variance of less than or equal to about 1.0, 0.9, 0.8, 0.7, 0.6, 0.5, 0.4, 0.3, 0.2, 0.15, or 0.1.

Aspect 7 pertains to the bioreactor system of any one of Aspects 1-6, wherein the cell culture matrix is arranged in a fixed bed configuration in the interior reservoir.

Aspect 8 pertains to the bioreactor system of Aspect 7, wherein the uniform flow is maintained through a bed height of the fixed bed, the bed height being greater than or equal to 10 cm; 20 cm; 30 cm; 40 cm; 50 cm; 60 cm; 70 cm; 80 cm; 90 cm; or 100 cm.

Aspect 9 pertains to the bioreactor system of any one of Aspects 1-8, wherein the curve of E(θ) has a single peak across all values of θ.

Aspect 10 pertains to the bioreactor system of Aspect 9, wherein the single peak is at a value of θ between 0.8 and 1.2; between 0.85 and 1.15; between 0.9 and 1.1; and between 0.95 and 1.05.

Aspect 11 pertains to the bioreactor system of any one of Aspects 1-10, wherein the cell culture matrix is configured for liquid media to flow through the cell culture matrix such that at least 90% of an entire liquid volume of the interior reservoir is replaced within 1.2 bioreactor volumes of liquid media flow through the interior reservoir.

Aspect 12 pertains to the bioreactor system of any one of Aspects 1-11, wherein the cell culture matrix is configured for liquid media to flow through the cell culture matrix such that a flow rate of the liquid media at any point along a width of the interior reservoir is within 15%, 10%, 5%, or 2% of the flow rate at any other point along the width, the flow rate being measured in a direction parallel to the direction from the inlet to the outlet.

Aspect 13 pertains to the bioreactor system of any one of Aspects 1-12, wherein the cell culture matrix is configured for liquid media to flow through the cell culture matrix such that a flow rate of the liquid media at any point within the cell culture matrix is within 15%, 10%, 5%, or 2% of the flow rate at any other point along within the cell culture matrix, the flow rate being measured in a direction parallel to the direction from the inlet to the outlet.

Aspect 14 pertains to the bioreactor system of any one of Aspects 1-13, wherein the cell culture matrix comprises a multilayer substrate, wherein each layer of the multilayer substrate comprises a physical structure and a porosity that are substantially regular and uniform.

Aspect 15 pertains to the bioreactor system of any one of Aspects 1-14, wherein the cell culture matrix comprises a substantially uniform porosity.

Aspect 16 pertains to the bioreactor system of any one of Aspects 1-16, wherein the uniform flow comprises laminar flow.

Aspect 17 pertains to the bioreactor system of any one of Aspects 1-16, wherein the reservoir is defined by a length and a width, the length extending from a first end of the reservoir adjacent to the inlet to a second end of the reservoir adjacent to the outlet, the cell culture matrix having a width extending substantially across a width of the reservoir.

Aspect 18 pertains to the bioreactor system of any one of Aspects 14-17, wherein the multilayer substrate comprises a plurality of substrate disks stacked in the reservoir.

Aspect 19 pertains to the bioreactor system of Aspect 18, wherein the stack of the plurality of substrate disks is configured to exhibit substantially uniform fluid flow across a width of each of the plurality of substrate disks.

Aspect 20 pertains to the bioreactor system of Aspect 18, wherein multilayer substrate comprises a plurality of openings defining the porosity, the plurality of openings being arrayed in a regular or uniform pattern in each disk of the plurality of substrate disks.

Aspect 21 pertains to the bioreactor system of any one of Aspects 1-20, wherein the multilayer substrate comprises at least one of a molded polymer lattice, a 3D-printed lattice, and a woven substrate.

Aspect 22 pertains to the bioreactor system of Aspect 21, wherein the multilayer substrate comprises the woven substrate having a plurality of interwoven fibers with surfaces configured for adhering cells thereto, the plurality of interwoven fibers defining a plurality of openings.

Aspect 23 pertains to the bioreactor system of Aspect 22, wherein the cell culture matrix comprises a plurality of woven substrates, each woven substrate having a first side and a second side opposite the first side and separated from the first side by a thickness of the woven substrate.

Aspect 24 pertains to the bioreactor system of Aspect 23, wherein the plurality of woven substrates is arranged in a stack such that one of the first and second side of a substrate is facing the first or second side of an adjacent substrate.

Aspect 25 pertains to the bioreactor system of Aspect 23 or Aspect 24, wherein at least a portion of the plurality of substrates are not separated by a spacer material or barrier, or are in physical contact with each other.

Aspect 26 pertains to the bioreactor system of Aspect 22, wherein the cell culture matrix is disposed in the bioreactor vessel such that a bulk flow direction of media through the bioreactor vessel is perpendicular to the first and second sides.

Aspect 27 pertains to the bioreactor system of any one of Aspects 1-26, wherein the cell culture matrix exhibits uniform fluid flow across at least 90% of a surface area of the cell culture matrix.

Aspect 28 pertains to the bioreactor system of any one of Aspects 1-27, wherein the cell culture matrix exhibits plug flow through a length of the cell culture matrix in a direction of fluid flow.

Aspect 29 pertains to the bioreactor system of any one of Aspects 1-28, wherein the cell culture matrix is disposed in the interior reservoir at at least 85% occupancy, at least 90% occupancy, at least 95% occupancy, at 100% or greater occupancy, at 102% or greater occupancy, at 105% or greater occupancy, or at 110% or greater occupancy.

Aspect 30 pertains to the bioreactor system of any one of Aspects 1-29, wherein the cell culture matrix is disposed in the interior reservoir in a compressed packing configuration.

Aspect 31 pertains to a bioreactor system for culturing cells, the system comprising: a cell culture vessel comprising at least one interior reservoir, an inlet fluidly connected to the reservoir, and an outlet fluidly connected to the reservoir; and a cell culture matrix disposed in the reservoir, the cell culture matrix comprising a substrate configured for adhering cells thereto, wherein the cell culture matrix is configured to maintain a uniform flow of liquid media through the cell culture matrix, such that E(θ) is less than or equal to 0.2 or less than or equal to 0.1, when θ is less than or equal to 0.8, 0.75, 0.7, 0.6, or 0.5.

Aspect 32 pertains to the bioreactor system of Aspect 31, wherein E(θ) is less than or equal to 0.2 or less than or equal to 0.1, when θ is greater than or equal to 1.2, 1.25, 1.3, 1.4, or 1.5.

Aspect 33 pertains to the bioreactor system of Aspect 31 or Aspect 32, wherein E′(θ)=0 at a value of θ between 0.6 and 1.4; between 0.7 and 1.3; between 0.8 and 1.2; and between 0.9 and 1.1.

Aspect 34 pertains to the bioreactor system of any one of Aspects 31-33, wherein the cell culture matrix is configured to maintain the uniform flow of liquid media through the cell culture matrix with a normalized variance of less than or equal to about 1.0, 0.9, 0.8, 0.7, 0.6, 0.5, 0.4, 0.3, 0.2, 0.15, or 0.1.

Aspect 35 pertains to the bioreactor system of any one of Aspects 31-34, wherein the cell culture matrix comprises a multilayer substrate, wherein each layer of the multilayer substrate comprises a physical structure and a porosity that are substantially regular and uniform.

Aspect 36 pertains to the bioreactor system of any one of Aspects 31-35, wherein the cell culture matrix comprises a substantially uniform porosity.

Aspect 37 pertains to the bioreactor system of any one of Aspects 31-36, wherein the uniform flow comprises laminar flow.

Aspect 38 pertains to the bioreactor system of any one of Aspects 31-37, wherein the reservoir is defined by a length and a width, the length extending from a first end of the reservoir adjacent to the inlet to a second end of the reservoir adjacent to the outlet, the cell culture matrix having a width extending substantially across a width of the reservoir.

Aspect 39 pertains to the bioreactor system of any one of Aspects 35-38, wherein the multilayer substrate comprises a plurality of substrate disks stacked in the reservoir.

Aspect 40 pertains to the bioreactor system of Aspect 39, wherein the stack of the plurality of substrate disks is configured to exhibit substantially uniform fluid flow across a width of each of the plurality of substrate disks.

Aspect 41 pertains to the bioreactor system of Aspect 39, wherein multilayer substrate comprises a plurality of openings defining the porosity, the plurality of openings being arrayed in a regular or uniform pattern in each disk of the plurality of substrate disks.

Aspect 42 pertains to the bioreactor system of any one of Aspects 31-41, wherein the multilayer substrate comprises at least one of a molded polymer lattice, a 3D-printed lattice, and a woven substrate.

Aspect 43 pertains to the bioreactor system of Aspect 42, wherein the multilayer substrate comprises the woven substrate having a plurality of interwoven fibers with surfaces configured for adhering cells thereto, the plurality of interwoven fibers defining a plurality of openings.

Aspect 44 pertains to the bioreactor system of Aspect 43, wherein the cell culture matrix comprises a plurality of woven substrates, each woven substrate having a first side and a second side opposite the first side and separated from the first side by a thickness of the woven substrate.

Aspect 45 pertains to the bioreactor system of Aspect 44, wherein the plurality of woven substrates is arranged in a stack such that one of the first and second side of a substrate is facing the first or second side of an adjacent substrate.

Aspect 46 pertains to the bioreactor system of Aspect 44 or Aspect 45, wherein at least a portion of the plurality of substrates are not separated by a spacer material or barrier, or are in physical contact with each other.

Aspect 47 pertains to the bioreactor system of Aspect 43, wherein the cell culture matrix is disposed in the bioreactor vessel such that a bulk flow direction of media through the bioreactor vessel is perpendicular to the first and second sides.

Aspect 48 pertains to the bioreactor system of any one of Aspects 31-47, wherein the cell culture matrix exhibits uniform fluid flow across at least 90% of a surface area of the cell culture matrix.

Aspect 49 pertains to the bioreactor system of any one of Aspects 31-48, wherein the cell culture matrix exhibits plug flow through a length of the cell culture matrix in a direction of fluid flow.

Aspect 50 pertains to the bioreactor system of any of Aspects 31-49, wherein the cell culture matrix is disposed in the interior reservoir at at least 85% occupancy, at least 90% occupancy, at least 95% occupancy, at 100% or greater occupancy, at 102% or greater occupancy, at 105% or greater occupancy, or at 110% or greater occupancy.

Aspect 51 pertains to the bioreactor system of any one of Aspects 31-50, wherein the cell culture matrix is disposed in the interior reservoir in a compressed packing configuration.

Aspect 52 pertains to a bioreactor system for culturing cells, the system comprising: a cell culture vessel comprising at least one interior reservoir, an inlet fluidly connected to the reservoir, and an outlet fluidly connected to the reservoir; and a cell culture matrix disposed in the reservoir, the cell culture matrix comprising a substrate configured for adhering cells thereto, wherein the cell culture matrix is disposed in the interior reservoir at at least 85% occupancy, at least 90% occupancy, at least 95% occupancy, at 100% or greater occupancy, at 102% or greater occupancy, at 105% or greater occupancy, or at 110% or greater occupancy.

Aspect 53 pertains to the bioreactor system of Aspect 52, wherein the cell culture matrix is disposed in the interior reservoir in a compressed packing configuration.

### Definitions

“Wholly synthetic” or “fully synthetic” refers to a cell culture article, such as a microcarrier or surface of a culture vessel, that is composed entirely of synthetic source materials and is devoid of any animal derived or animal sourced materials. The disclosed wholly synthetic cell culture article eliminates the risk of xenogeneic contamination.

“Include,” “includes,” or like terms means encompassing but not limited to, that is, inclusive and not exclusive.

“Users” refers to those who use the systems, methods, articles, or kits disclosed herein, and include those who are culturing cells for harvesting of cells or cell products, or those who are using cells or cell products cultured and/or harvested according to embodiments herein.

“About” modifying, for example, the quantity of an ingredient in a composition, concentrations, volumes, process temperature, process time, yields, flow rates, pressures, viscosities, and like values, and ranges thereof, or a dimension of a component, and like values, and ranges thereof, employed in describing the embodiments of the disclosure, refers to variation in the numerical quantity that can occur, for example: through typical measuring and handling procedures used for preparing materials, compositions, composites, concentrates, component parts, articles of manufacture, or use formulations; through inadvertent error in these procedures; through differences in the manufacture, source, or purity of starting materials or ingredients used to carry out the methods; and like considerations. The term “about” also encompasses amounts that differ due to aging of a composition or formulation with a particular initial concentration or mixture, and amounts that differ due to mixing or processing a composition or formulation with a particular initial concentration or mixture.

“Optional” or “optionally” means that the subsequently described event or circumstance can or cannot occur, and that the description includes instances where the event or circumstance occurs and instances where it does not.

The indefinite article “a” or “an” and its corresponding definite article “the” as used herein means at least one, or one or more, unless specified otherwise.

Abbreviations, which are well known to one of ordinary skill in the art, may be used (e.g., “h” or “hrs” for hour or hours, “g” or “gm” for gram(s), “mL” for milliliters, and “rt” for room temperature, “nm” for nanometers, and like abbreviations).

Specific and preferred values disclosed for components, ingredients, additives, dimensions, conditions, and like aspects, and ranges thereof, are for illustration only; they do not exclude other defined values or other values within defined ranges. The systems, kits, and methods of the disclosure can include any value or any combination of the values, specific values, more specific values, and preferred values described herein, including explicit or implicit intermediate values and ranges.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that any particular order be inferred.

It will be apparent to those skilled in the art that various modifications and variations can be made without departing from the spirit or scope of the disclosed embodiments. Since modifications, combinations, sub-combinations and variations of the disclosed embodiments incorporating the spirit and substance of the embodiments may occur to persons skilled in the art, the disclosed embodiments should be construed to include everything within the scope of the appended claims and their equivalents.

1. A bioreactor system for culturing cells, the system comprising:
a cell culture vessel comprising at least one interior reservoir, an inlet fluidly connected to the reservoir, and an outlet fluidly connected to the reservoir; and
a cell culture matrix disposed in the reservoir, the cell culture matrix comprising a substrate configured for adhering cells thereto,
wherein the cell culture matrix is configured to maintain a uniform flow of liquid media through the cell culture matrix, such that the uniform flow satisfies the following expressions:
$${\int }_{θ 1}^{θ 2} E ( θ ) d θ = P ,$$
$$Δθ = {θ}_{2} - {θ}_{1} , and$$
$$Δθ \leq N ,$$
wherein P is greater than or equal to 0.7, and
wherein N is less than or equal to 0.5.

2. The bioreactor system of claim 1, wherein N is less than or equal to 0.5, and P is greater than or equal to 0.9.

3. The bioreactor system of claim 1, wherein E(θ) is less than or equal to 0.2, when θ is less than or equal to 0.8 and when θ is greater than or equal to 1.2.

4. (canceled)

5. The bioreactor system of claim 1, wherein E′(θ)=0 at a value of θ between 0.8 and 1.2.

6. The bioreactor system of claim 1, wherein the cell culture matrix is configured to maintain the uniform flow of liquid media through the cell culture matrix with a normalized variance of less than or equal to about 0.9.

7. The bioreactor system of claim 1, wherein the cell culture matrix is arranged in a fixed bed configuration in the interior reservoir.

8. The bioreactor system of claim 7, wherein the uniform flow is maintained through a bed height of the fixed bed, the bed height being greater than or equal 20 cm.

9. The bioreactor system of claim 1, wherein the curve of E(θ) has a single peak across all values of θ.

10. The bioreactor system of claim 9, wherein the single peak is at a value of θ between 0.8 and 1.2.

11. The bioreactor system of claim 1, wherein the cell culture matrix is configured for liquid media to flow through the cell culture matrix such that at least 90% of an entire liquid volume of the interior reservoir is replaced within 1.2 bioreactor volumes of liquid media flow through the interior reservoir.

12. (canceled)

13. The bioreactor system of claim 1, wherein the cell culture matrix is configured for liquid media to flow through the cell culture matrix such that a flow rate of the liquid media at any point within the cell culture matrix is within 15%, 10%, 5%, or 2% of the flow rate at any other point along within the cell culture matrix, the flow rate being measured in a direction parallel to the direction from the inlet to the outlet.

14. The bioreactor system of claim 1, wherein the cell culture matrix comprises a multilayer substrate, wherein each layer of the multilayer substrate comprises a physical structure and a porosity that are substantially regular and uniform. 15-26. (Canceled)

27. The bioreactor system of claim 1, wherein the cell culture matrix exhibits uniform fluid flow across at least 90% of a surface area of the cell culture matrix.

28. The bioreactor system of claim 1, wherein the cell culture matrix exhibits plug flow through a length of the cell culture matrix in a direction of fluid flow.

29. The bioreactor system of claim 1, wherein the cell culture matrix is disposed in the interior reservoir at 95% or greater occupancy.

30. The bioreactor system of claim 1, wherein the cell culture matrix is disposed in the interior reservoir in a compressed packing configuration.

31. A bioreactor system for culturing cells, the system comprising:
a cell culture vessel comprising at least one interior reservoir, an inlet fluidly connected to the reservoir, and an outlet fluidly connected to the reservoir; and
a cell culture matrix disposed in the reservoir, the cell culture matrix comprising a substrate configured for adhering cells thereto, wherein the cell culture matrix is configured to maintain a uniform flow of liquid media through the cell culture matrix, such that E(θ) is less than or equal to 0.2 when θ is less than or equal to 0.8.

32. (canceled)

33. (canceled)

34. The bioreactor system of claim 31, wherein the cell culture matrix is configured to maintain the uniform flow of liquid media through the cell culture matrix with a normalized variance of less than or equal to about 0.9.

35-51. (canceled)

52. A bioreactor system for culturing cells, the system comprising:
a cell culture vessel comprising at least one interior reservoir, an inlet fluidly connected to the reservoir, and an outlet fluidly connected to the reservoir; and
a cell culture matrix disposed in the reservoir, the cell culture matrix comprising a substrate configured for adhering cells thereto,
wherein the cell culture matrix is disposed in the interior reservoir at at least 85% occupancy, at least 90% occupancy, at least 95% occupancy, at 100% or greater occupancy, at 102% or greater occupancy, at 105% or greater occupancy, or at 110% or greater occupancy.

53. The bioreactor system of claim 52, wherein the cell culture matrix is disposed in the interior reservoir in a compressed packing configuration.
